(19) United States
(12) Patent Application Publication
(10) Pub. No.: US 2019/0072562 A1
(43) Pub. Date: Mar. 7, 2019

(54) SYSTEM AND METHOD FOR DETERMINATION OF LIGAND-TARGET BINDING BY MULTI-PHOTON FLUORESCENCE ANISOTROPY MICROSCOPY

(71) Applicant: THE GENERAL HOSPITAL CORPORATION, Boston, MA (US)

(72) Inventors: Claudio Vinegoni, Cambridge, MA (US); Ralph Weissieder, Boston, MA (US); J. Matthew Dubach, Boston, MA (US); Ralph Mazitschek, Belmont, MA (US)

(21) Appl. No.: 16/179,177

(22) Filed: Nov. 2, 2018

Related U.S. Application Data
(63) Continuation of application No. 15/305,305, filed on Oct. 19, 2016, now abandoned.
(60) Provisional application No. 61/982,551, filed on Apr. 22, 2014.

(51) Int. Cl. G01N 33/58; G02B 21/18; G02B 21/16; G02B 21/00; G01N 21/64; A61B 5/00

(52) U.S. Cl. CPC G01N 33/582; G02B 21/18; G02B 21/16; G02B 21/0096; G01N 21/6458; G01N 2021/6439; G01N 21/6445; G01N 21/6428; G01N 21/6408; A61B 5/0075; A61B 5/0071

(57) ABSTRACT

A multiphoton fluorescence anisotropy microscopy live cell imaging system and method to measure and map drug-target interaction in real time at subcellular resolution. Proposed modality enables a direct measurement of drug/target binding in vivo, high-resolution spatial and temporal mapping of bound and unbound drug distribution, and presents an versatile tool to enhance understanding of drug activity. Application of the system to measurement of intracellular target engagement of the chemotherapeutic Olaparib, a poly(ADP-ribose) polymerase inhibitor, in live cells and within a tumor in vivo.

### CROSS-REFERENCE TO RELATED APPLICATIONS

The present application is a continuation of the U.S. patent application Ser. No. 15/305,305, published as US 2017/0045521, which is a national phase of the international patent application No. PCT/US15/27052 filed on Apr. 22, 2015, which in turn claims priority from the U.S. Provisional Patent Application No. 61/982,551, titled “Multi-Photon Fluorescence Anisotropy Microscopy” and filed on Apr. 22, 2014. The disclosure of each of the above-identified patent applications is incorporated by reference herein.

### STATEMENT REGARDING FEDERALLY SPONSORED RESEARCH OR DEVELOPMENT

This invention was made with government support with grant under contract No. HHSN268201000044C awarded by the National Heart, Lung and Blood Institute, National Institute of Health, Department of Health and Human Services, grant nos. T32CA079443 and P50CA086355 awarded by the National Cancer institute, and grant no. R01EB006432 awarded by the institute of Biomedical Engineering. The Government has certain rights in the invention.

### TECHNICAL FIELD

The present invention generally relates to fluorescent microscopy and, more particularly, to a multi-photon fluorescent microscopy system and method for visualizing and measuring a degree of ligand-target interaction in real time at the cellular level.

### BACKGROUND

Small molecule therapeutic drugs typically exert their effects through binding to one or a few protein targets. This critical interaction—a prerequisite of therapeutic drug efficacy—is often insufficiently understood and generally cannot be visualized in live cells or entire organisms due to the lack of methods to directly measure drug target engagement in a biological setting. As a result, most of available knowledge about the subject is incomplete, as such knowledge relies on target extraction assay systems or indirect measurements (during which critical spatiotemporal information is lost). Clearly, the status quo complicates further drug development.

The critical interaction between small molecules and targets can also be visualized and measured not only in a direct way but also in indirect way in competitive mode for example competing with the molecule of interest.

Recent advances in chemical techniques have allowed the creation of fluorescent drugs, prodrugs and activity based probes to interrogate target engagement. To date, most of these compounds have been used in vitro, while a select few have been used in vivo for imaging drug distribution (pharmacokinetics) or tumor detection. However, to realize the full potential of intravital imaging with fluorescently-labeled compounds determination of target engagement with subcellular resolution is needed.

### SUMMARY

An embodiment of the present invention provides a system for spatially and/or temporally resolving a portion of a target (or a whole target) containing target-bound fluorescent or fluorescently labeled molecules or ligands. (For the purposes of the present invention, the term “ligand” refers to a small molecule that can be imparted with fluorescent properties. Ligands can include small molecules with pharmaceutical activity or derivatives. Targets include but are not limited to biomacromolecules such as peptides, proteins, carbohydrates, lipids, nucleic acids, for example.) Such system includes a source of light unit configured to generate light to be absorbed by a fluorescent or fluorescently labeled molecule, such as, for example, a fluorescently labeled drug via a multi-photon process; and an optical system positioned to optically relay light generated by the source of light unit onto an object plane of the system and form first and second images of the object plane (at first and second image planes respectively) in light emitted from the object plane such that a) the first image is formed in light emitted from the object plane and having only a first state of polarization, and b) the second image is formed in light emitted from the object plane and having only a second state of polarization; and a processor programmed to transform said first and second images into a third image representing spatial anisotropy of said target. In one implementation, the optical system includes a microscope configured to epi-collect said light emitted from the object plane; a first optical detector positioned to receive said light emitted from the object plane and having only the first state of polarization; and a second optical detector positioned to receive said light emitted form the object plane and having only the second state of polarization. In a specific case, the processor is programmed to calculate a spatial distribution of anisotropy of the target according to r=(I₁−I₂)/(I₁+2I₂), wherein r is a measure of said anisotropy, I₁ is the first image, and I₂ is the second image. In a related embodiment, the optical system includes a microscope configured to collect said light emitted from the object plane in a confocal mode; and the source of light unit is judiciously chosen to emit light sequentially at first and second polarization or to detect light at two detectors each positioned to receive light having a corresponding one of two different states of polarization.

Embodiments of the invention additionally provide a method for a spatially and/or temporally resolved optical detection of binding between fluorescently labeled molecules and a target. The method includes a step of optically imaging the target, in the presence of a fluorescently labeled compound, for example a fluorescently labeled drug, to form an image representing a degree of anisotropy of light emitted by the fluorescently labeled compound or drug. A step of optically imaging includes collecting light from the target with a microscopy system (configured as a wide-angle epi-microscopy system or a confocal system). Alternatively or in addition, the step of optical imaging includes determining of lifetime of fluorescence emitted by the fluorescently labeled compound or drug which can be bound to at least a portion of its target(s). Alternatively or in addition, the step of optically imaging includes forming first and second images with first and second optical detectors, respectively, in fluorescent light emitted by the target. In a related implementation, the method additionally comprises causing the fluorescently labeled compound or drug to generate the fluorescent light by exciting it with a multi-photon process and/or acquiring said fluorescent light having only a first state of polarization with the first optical detector, acquiring said fluorescent light having only a second state of polarization with the second optical detector. A specific embodiment of the method also includes a step of calculating spatial distribution of anisotropy of the target according to r=(I₁−I₂)/(I₁+2I₂), wherein r is a measure of the anisotropy of the target, and I₁ denotes the first image and I₂ denotes the second image.

### BRIEF DESCRIPTION OF THE DRAWINGS

The invention will be more fully understood by referring to the following Detailed Description of Specific Embodiments in conjunction with the not-to scale Drawings, of which:

FIG. 1A is a schematic representation of the two-photon photoselection process in a randomly oriented distribution of fluorophores and the resulting fluorescence emission for low (isotropic) and high (anisotropic) rotational correlation times (τθ).

FIG. 1B is a diagram of the optical setup of the multiphoton fluorescence anisotropy microscope system according to an embodiment of the invention.

FIG. 1C illustrates anisotropy and fluorescence intensity images. Intensity (A and C) and corresponding anisotropy (B and D) images of a fluorescent microscope slide measured at two different laser excitation powers.

FIG. 2A is a plot illustrating the Me5-BODIPY anisotropy dependence on viscosity, as measured in glycerol with an embodiment of the disclosure.

FIG. 2B illustrates distribution of fluorescent optical power among two orthogonal states of polarization with a scale bar 20 μm.

FIG. 2C illustrates anisotropy as measured by MFAM and compared to single photon plate reader measurements.

FIG. 2D illustrates anisotropy artifacts present at the border of the field-of-view.

FIG. 2E illustrates anisotropy within the objective field of view.

FIG. 3A provides representations of optical characterization of an embodiment of the MFAM system of the invention, for MFAM point spread function characterization.

FIG. 3B also provides representations of optical characterization of an embodiment of the MFAM system of the invention, for MFAM point spread function characterization.

FIG. 3C also provides representations of optical characterization of an embodiment of the MFAM system of the invention, for MFAM point spread function characterization.

FIG. 3D also provides representations of optical characterization of an embodiment of the MFAM system of the invention, for MFAM point spread function characterization.

FIG. 3E is am image showing two highly homogeneous populations of green fluorescent microspheres with distinct anisotropy values suspended in a 2% agarose solution.

FIG. 4A is a schematic illustrating the anisotropy value of Biotin-BODIPY (mw 676.62) increases as a function of binding to NeutrAvidin (mw 60 kDa) (filled triangles), which is suppressed in the presence of 10× unlabeled biotin as competitor (open triangles).

FIG. 4B is a schematic showing Average±stdev anisotropy of non-specifically interacting (light gray) and PARP bound (dark gray) AZD2281-BODIPY FL (n=3).

FIG. 4C is a 3D anisotropy image and corresponding planar and axial cross sections of live HT1080 cells loaded with AZD2281-BODIPY FL, where light gray, corresponds to fluorescent drug molecules that are non-specifically bound and dark gray corresponds to fluorescent drug molecules with high anisotropy suggesting target (PARP) binding. Normal fluorescence images are shown in FIG. 18. Scale bar: 16 microns.

FIG. 4D is a 3D anisotropy image and corresponding planar and axial cross sections of live HT1080 cells loaded with AZD2281-BODIPY FL and washed for 30 minutes. Scale bars: 20 microns.

FIG. 5A is a set of images of target engagement over time, showing anisotropy and corresponding fluorescence images of AZD2281-BODIPY FL at four representative time points during drug loading and after washing.

FIG. 5B is a set of images of target engagement over time, showing anisotropy and corresponding fluorescence images of AZD2281-BODIPY FL at four representative time points during drug loading and after washing, in a manner similar to FIG. 5A, but in the presence of 5 fold higher concentration of unlabeled AZD2281 (competition). Scale bars: 20 microns.

FIG. 6A is a set of graphs showing real time imaging of drug target engagement in live cells, for values measured in the cytoplasmic region of the cells.

FIG. 6B is a set of graphs showing real time imaging of drug target engagement in live cells, for values measured in the nuclear region of the cells.

FIG. 7A is an in vivo fluorescence image of injected fluorescent microspheres (light gray) in the vascularized (dark gray) tissue fascia of a mouse dorsal skinfold window chamber. Scale bar: 50 microns.

FIG. 7B is a graph showing anisotropy of the injected fluorescent microspheres as a function of depth within the tissue fascia. Each point corresponds to a single bead measurements.

FIG. 7C is a confocal fluorescence image of HT1080 H2B mApple cells (dark gray) in a mouse dorsal skinfold window chamber. After 1-2 weeks, the tumor area is highly vascularized and, upon intravenous injection, perfused with AZD2281-BODIPY FL (light gray). The white square indicates the imaged area in FIG. 7D. Scale bar: 100 microns.

FIG. 7D is a set of images, including in vivo anisotropy (top) images and fluorescence (bottom) images of AZD2281-BODIPY FL following intravenous infusion (left) and 34 minutes later (right). Scale bar: 20 microns.

FIG. 7E is a graph showing overall image intensity (black), nuclear intensity (gray) and nuclear anisotropy (unfilled, striped) as measured from the images in FIG. 7D. Nuclear intensity and anisotropy values are average±std error (n=90 for image t1, n=102 for image t1+34 min). Fluorescence intensity refers to the sum of both perpendicular and parallel channels.

FIG. 8A is a graph that illustrates a fundamental limit of anisotropy resolution based on number of photons detected. Intensity (circles) and absolute value of percent change in anisotropy (squares) as a function of excitation power. At low excitation power, the low SNR of the detected intensity affects anisotropy determination. The lower the detected intensity, the higher will be the error on the anisotropy determination due to the proximity of the signal to the noise level. Measurements were done on fluorescent microscope slide with an anisotropy value of 0.34. The noise level is equal to 200 a.u. (light arrow). For recorded intensities below 280 a.u (horizontal line) the calculated value of anisotropy differs 10% at most from the anisotropy value calculated at higher intensities (dark arrow).

FIG. 8B is a set of images and graphs that illustrate an anisotropy profile of a single fluorescent microsphere. (A) Anisotropy image of a horizontal plane optically sectioned through the agare sample of FIG. 1E. Box expanded into (B). Scale bar 20 μm. (B) Enlarged anisotropy image of a single microsphere. Intensity (black circles) and anisotropy (squares) profiles along the two orthogonal white lines are plotted. The anisotropy remains constant along the microsphere profile.

FIG. 9A shows two populations of fluorescent microspheres. (A) Two populations of six micron green-fluorescent microspheres with discrete values of fluorescence intensity (100% and 30% respectively) were used (FIG. 3a). The fluorescence intensity of the microspheres in each suspension is highly homogeneous. Due to homo-FRET the two distinct populations of microspheres (100% and 30%) present two different values of anisotropy each one highly homogeneously distributed. Fluorescent (left) and anisotropy (right) images of the two populations (30% top, 100% bottom) of fluorescent microscopheres are shown. The population with low fluorescence intensity (top) has a high value of anisotropy (0.274±0.008). While, the population with high fluorescence intensity (bottom) present a low value of anisotropy (0.193±0.005), average±stdev. (B) Scatter plot of anisotropy as function of intensity for the two microspheres populations. As clearly evident the two populations are significantly separated in both intensity and anisotropy. The average (single circles) and distribution (black circles) of each population are shown on the right. Scale bar 20 μm.

FIG. 9B shows FLIM images and lifetime measurements of fluorescent microspheres FIG. 9A with 100% (A) and 30% (B) relative intensity.

FIG. 10 illustrates anisotropy of AZD2281-BODIPY FL and colocalization of AZD2281-BODIPY FL with PARP expression. (A) Extension of FIG. 2B, showing the anisotropy of AZD2281-BODIPY FL in DMSO alone (dark gray) compared to non-specific binding to FBS (light gray) and binding to PARP in the presence of FBS (black). Data are average±stdev (n=3). (B) Fluorescence signal of AZD2281-BODIPY FL (left) colocalizes with PARP expression as evidenced by anti-PARP immunofluorescence (right). Fluorescence data in panel B are from Adibekian et al (J. AM. Chem. Soc., 134, 10345-10348, 2012).

FIG. 11 illustrates anisotropy in the presence and absence of H2B mApple labeling. Confocal (AZD2281-BODIPY FL and mApple fluorescence channels) and multiphoton (AZD2281-BODIPY FL fluorescence and anisotropy) images of HT1080 cells. (A), HT1080 cells expressing H2B mApple loaded with AZD2281-BODIPY FL. (B), HT1080 cells expressing H2B mApple loaded with AZD2281-BFL and washed. (C), HT1080 cells loaded with AZD2281-BODIPY FL. (D), HT1080 cells loaded with AZD2281-BODIPY FL and washed. (E), HT1080 cells with no drug present. (F), HT1080 cells expressing H2B mApple with no drug present. Intensity and anisotropy scale bars apply to all images. Scale bar 20 μm.

FIG. 12 illustrates anisotropy of AZD2281-BODIPY FL in different cell types: Anisotropy and multiphoton fluorescence images of three different cell lines loaded with AZD2281-BODIPY FL and after washing. (A) HCC1937, (B) MHH-ES-1 and (C) MDA-MD-436 cells. Scale bars: A, 30 μm; B,C, 20 μm.

FIG. 13 provides illustration to free BODIPY loading in HT1080 cells. Fluorescence (top) and anisotropy (bottom) images of HT1080 cells loaded and washed, with (A) Me5-BODIPY and AZD2281-BODIPY FL, (B) Me5-BODIPY only and (C) AZD2281-BODIPY FL only. In (B) dashed line indicates the nuclei. Scale bar 20 μm.

FIG. 14 presents FLIM images of HT1080 cells loaded with AZD2281-BODIPY FL. HT1080 cells loaded with AZD2281-BODIPY FL and imaged 1 min after washing. (A) Fluorescence intensity, (B) FLIM image. (C) Intracellular fluorescence lifetime within the nucleus and the cytoplasm (n=28 cells, over 5 experiments; average±stdev).

FIG. 15 is a plot illustrating intracellular percentage of bound AZD2281-BODIPY FL. The intracellular percent bound can be calculated for each measurement when the completely bound and unbound anisotropy values of AZD2281-BODIPY FL are known. The bound anisotropy value in the nucleus was determined after washing the cells over a period of 8 min to remove any unbound AZD2281-BODIPY FL. Competition measurements instead provide an unbound anisotropy value in the nucleus as none (or negligible amount) of the AZD2281-BODIPY FL is bound to the AZD2281 target. Therefore the percentage of specifically bound drug can be determined at any point using the measured value of anisotropy (FIGS. 5A, 5B). Points in the graph represent the average of the bound fraction of AZD2281-BODIPY FL in the nucleus of HT1080 cells over multiple time points following loading (n=4±stdev).

FIG. 16 present plots illustrating anisotropy dependency on depth as measured in tissue-phantoms. (A) Fluorescence intensity as a function of depth in diffusive tissue phantoms containing a uniform distribution of fluorescein and presenting different optical densities of respectively 2 (circles), 0.5 (squares), 0.25 (triangles), 0.1 (inverted triangles), 0.05 (black diamonds) and 0 (open circles). (B) Standard deviation of the calculated anisotropy value from the average value obtained in free solution (0.017) for all different tissue-phantoms of (A). (C) Imaging depth at which the standard deviation of the anisotropy is twice the value in free solution (black line in (B)), for phantoms with different optical densities (colors correspond to (A)).

FIG. 17 provides in vivo images of HT1080 H2B mApple cells. Colocalization of AZD2281-BODIPY FL two photon signal with the nuclei. Left, confocal fluorescence image of H2B mApple labeled nuclei of the HT1080 tumor cells as measured in vivo. Right, multiphoton fluorescence image of AZD2281-BODIPY FL of the same corresponding area. Scale bar 20 μm.

FIG. 18 provides fluorescence 3D reconstructions of drug engagement in vitro. In vitro 3D fluorescence (top) and anisotropy (bottom) image and corresponding planar and axial cross sections of HT1080 cells loaded with AZD2281-BODIPY FL. Figure compares with FIG. 2C,D. Scale bars: 16 μm and 20 μm.

FIG. 19 is a set of graphs illustrating anisotropy over time. A fluorescent microscope slide with an average anisotropy value of 0.28 was used as imaging sample. Anisotropy measurements of the same point in the fluorescent slide over a period of time of one hour are collected in order to test the stability of the imaging system due to temperature fluctuations. The percent change from the mean anisotropy value (A) fluctuates between +0.2% and −0.2%. (B) Percent change from the mean anisotropy measured daily for six days at different hours (without system recalibration), present a much higher degree of variation due to temperature changes related to centralized air conditioning (dashed lines indicate changes of +2% and −2%).

FIG. 20 is a flow-chart illustrating an embodiment of the method of the invention.

### DETAILED DESCRIPTION

The present invention stems from the realization that a specifically-modified fluorescence polarization methodology (FP) could be used to accurately measure drug binding in vitro and in vivo through multiphoton microscopy. Fluorescence polarization quantifies the degree of fluorescence depolarization with respect to the polarization excitation plane, providing insight into the state or environment of the excited fluorescent molecule. FP has been extensively used in non-imaging, plate reader and kinetic in vitro assays to measure numerous fluorescent molecule and molecular drug interactions including target engagement. Extending FP to optical microscopy imaging modalities could provide spatially- and temporally-resolved mapping, thereby enabling live cell imaging of target engagement of small molecule drugs. However, microscopy imaging methods based on FP have been more commonly used to study homo-FRET in membrane dynamics, structure in ordered biological systems and endogenous small molecules or labeled protein interactions.

This invention addresses the problem of insufficiency of intravital imaging with fluorescently-labeled compounds determination of target engagement having subcellular resolution by providing a multiphoton fluorescence anisotropy microscopy (MFAM) system and method to image intracellular drug-target binding distribution in vivo. With the use of the proposed system in conjunction with a specific drug candidate it was shown that the proposed modality is not only applicable to live cultured cells but also enables real-time imaging of drug-target engagement in vivo with submicron resolution.

For the purposes of this disclosure and accompanying claims, a real-time performance of a system is understood as performance which is subject to operational deadlines from a given event to a system's response to that event.

### System and Principle of Operation.

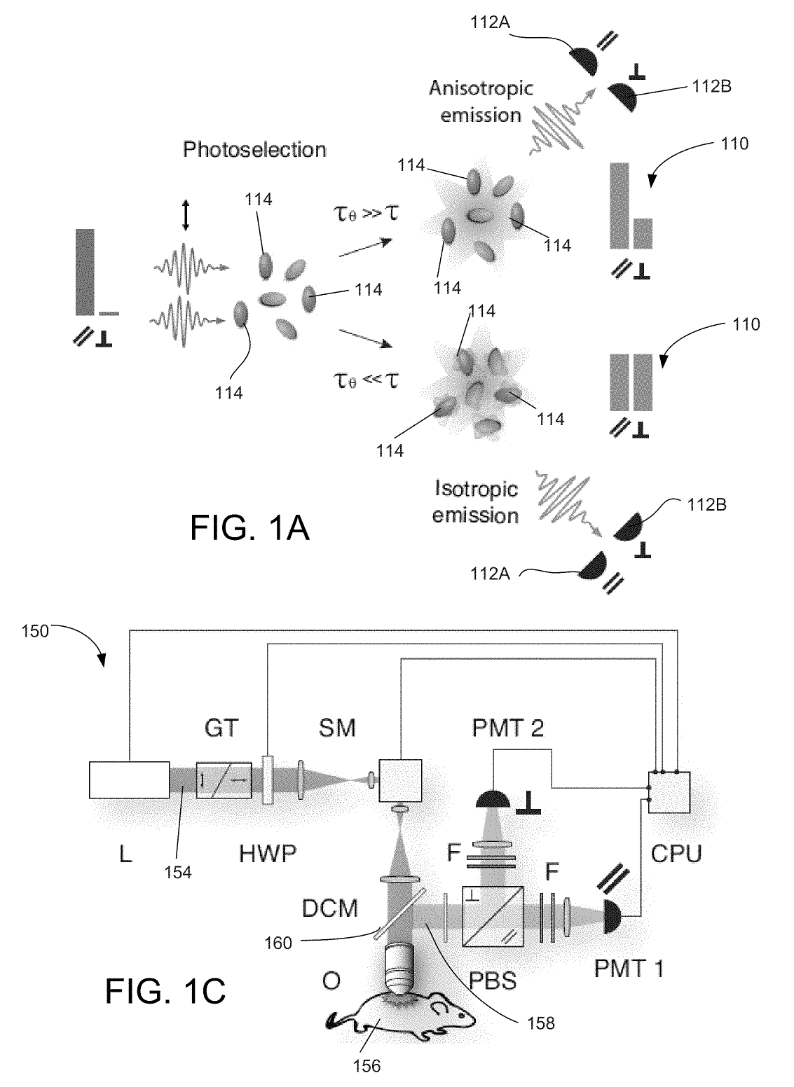

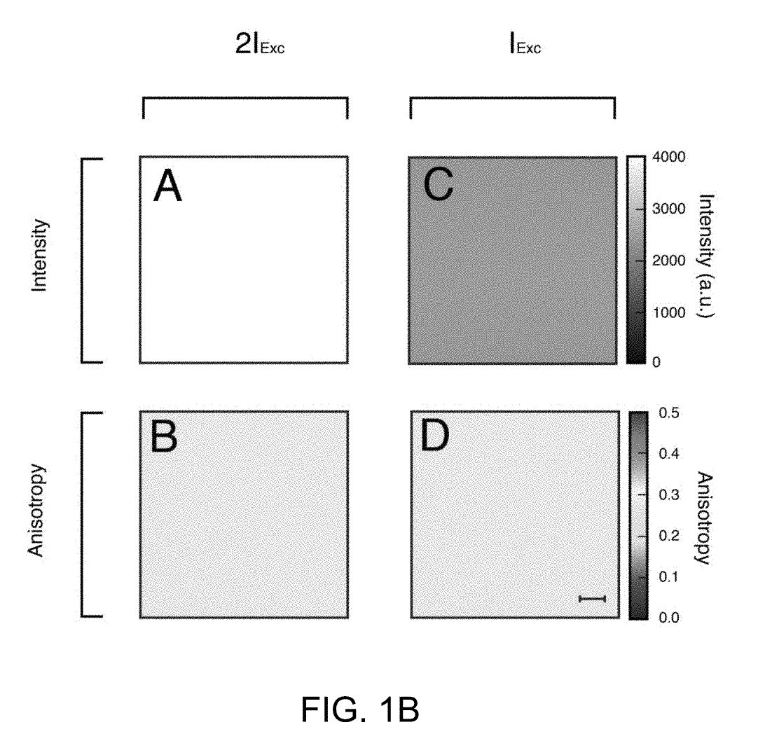

Following photoselection under polarized excitation, all excited fluorophores are aligned with the same emission dipole orientation. However, due to the presence of rotational Brownian motion, fluorophores rotate with a correlation time τ_{θ}, that is dependent on viscosity, molecule size and temperature. If the excited fluorophore is free to rapidly rotate on a timescale that is shorter than its fluorescence lifetime τ (τ_{θ}<<τ), the emission will be isotropic (and therefore depolarized). However, during the slow rotation, the rotational correlation time increases (τ_{θ}>>τ) and emission is preferentially aligned along one axis, as shown in FIG. 1A. In FIG. 1A, bars 110 indicate schematically the distribution of emission along the two orthogonal linear polarization components (∥, ⊥) as measured at the two detectors, 112A, 112B, for the two cases. Dark elongated ellipsoid 114 represent excited molecules.

Furthermore, a change in the fluorescence lifetime also effects the state of polarization of the emitted light, because molecules have less or more time to rotate before the act of emission. To characterize the extent of linearly polarized emission, fluorescence anisotropy (FA), a dimensionless parameter similar to FP and independent of excitation intensity can be calculated, such as illustrated in FIG. 1B. In particular, FIG. 1B illustrates anisotropy and fluorescence intensity images. Intensity (A and C) and corresponding anisotropy (B and D) images of a fluorescent microscope slide measured at two different laser excitation powers. Scale bar 20 μm.

According to the idea of the invention, the results of measurements of anisotropy are used to assess the rotational diffusion rate of molecules which, in turn, is further used to directly assess engagement of drug with the target. The use of multiphoton microscopy to determine a degree of anisotropy of an object (such as a biological tissue, or a fluorescently labeled drug) offers several advantages over other imaging modalities. Extended light penetration depth enables relatively deep imaging in tissues in a physiologically relevant context, while a diminished scattering component in the near-infrared (NIR) reduces scattering of light in the tissue. Therefore, multiphoton microscopy, with its low phototoxicity and high axial resolution, is ideally suited for high-resolution drug target interaction imaging within single cells, in vitro and in tissue.

An example of the system and method of the MFAM imaging, configured according to the idea of the invention, may utilize a custom-adapted commercial unit, as shown in FIG. 1C. In this example, the optical setup 150 is based on a custom modified Olympus FV1000-MPE (Olympus, USA) laser scanning microscopy system equipped with an upright BX61-WI microscope (Olympus, USA). Excitation light (dark gray beam, 154) from a Ti:sapphire laser, L, was filtered with the Glan-Thompson prism, GT, to select a linear state of polarization and then focused onto the imaged sample 156 with a 25×1.05 NA water immersion objective, O (XLPlan N, 2 mm working distance, Olympus). Fluorescent light emitted by the sample 156 (light gray beam, 158) was epi-collected, separated into two linearly orthogonally-polarized components with the use of a polarization beam splitter (PBS), and spectrally filtered with the optical filters, F, before non-descanned detection with optical detectors (in this non-limiting example—photomultiplier tubes, PMT1 and PMT2). In a related embodiment, a modified configuration of the system can be used. For example both filters F could be removed and substituted by only one filter G placed before the polarization beam splitter (PBS). The optical imaging data were processed with the use of a programmable computer processor, CPU. The MaiTai DeepSee Ti:sapphire pulsed laser (Spectra Physics) had a pulse-width of 110 fs and a repetition rate of 80 MHz. Laser was tuned at 910 nm for a two-photon excitation of pentamethyl (Me5)-BODIPY and BODIPY FL.

In further reference to FIG. 1C, fluorescence emission was detected in epi-collection mode through the same focusing objective. A dichroic filter 160 (690 nm) diverted the fluorescent light toward a non-descanned detection path, followed by a low pass filter (685 nm). Along the detection path a polarizing beam splitter, PBS (Edmund optics) was inserted to separate the light in two orthogonal states of polarization, each one followed by a bandpass filter F (490-540 nm, Chroma). Light portions having two orthogonal state of linear polarization were then focused and detected by two different photodetectors (each detecting light in only one polarization state (marked as I/∥, I⊥). Light 154 exciting the sample excitation light was linearly polarized. Other different state(s) of polarization can be chosen. A dual-detector acquisition may be advantageous in some embodiments to avoid severe anisotropy artifacts induced by fluctuations of intensity of the excitation light 154. A dual-detector acquisition system can also replaced by a single detector acquisition. If this is the case two separate images need to be collected. Each one at different orthogonal states of polarization.

In a related embodiment, the imaging system of the invention acquires fluorescent light using only one photodetector, and the polarization state is selected by acting respectively on an optical element such as a waveplate, a polarization beamsplitter, or a polarization filter.

In a related embodiment, the imaging system of the invention was also configured to operate as a confocally imaging system. In this embodiment, linearly polarized light excites a fluorescently labeled molecule and fluorescent light is detected by two photodetectors each acquiring only light with a corresponding one of two orthogonal states of polarization.

In a related embodiment, a serial 2D imaging was carried out to generate a sequence of 2D images of the sample in fluorescent light to form a 3D representation of spatial distribution of the regions of tissue to which identified molecules were bound. Such 3D representation was effectuated with equipping a microscope objective with a Z-axis motor (with a 0.01 μm step size). Different areas along the entire size of the dorsal window chamber were sequentially imaged over time using a microscope-controlled long-range XY-axis translation stage. Also the same strategy was applied to acquire 3D representation of cells in vitro.

### System Test.

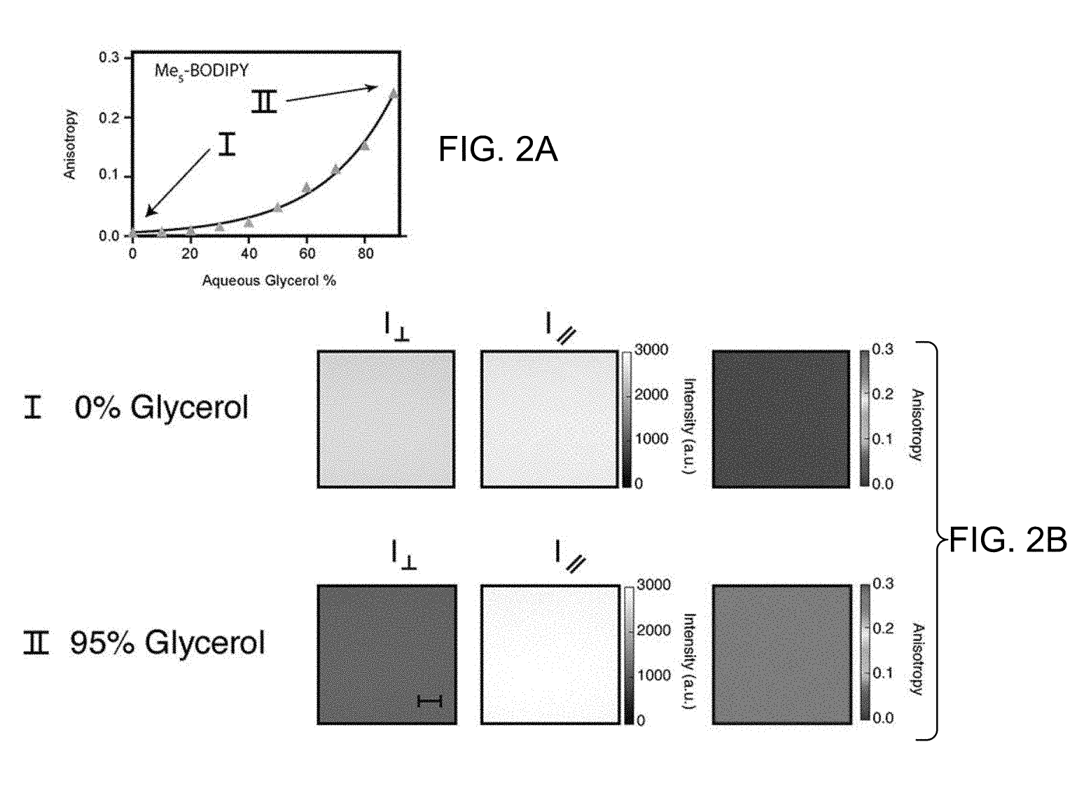

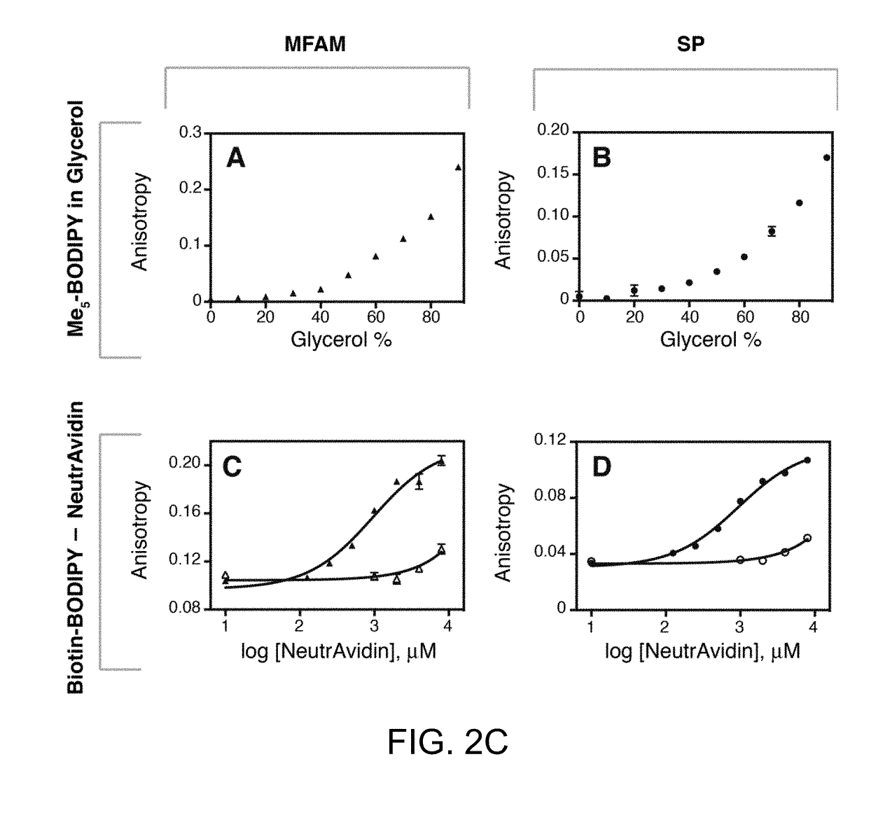

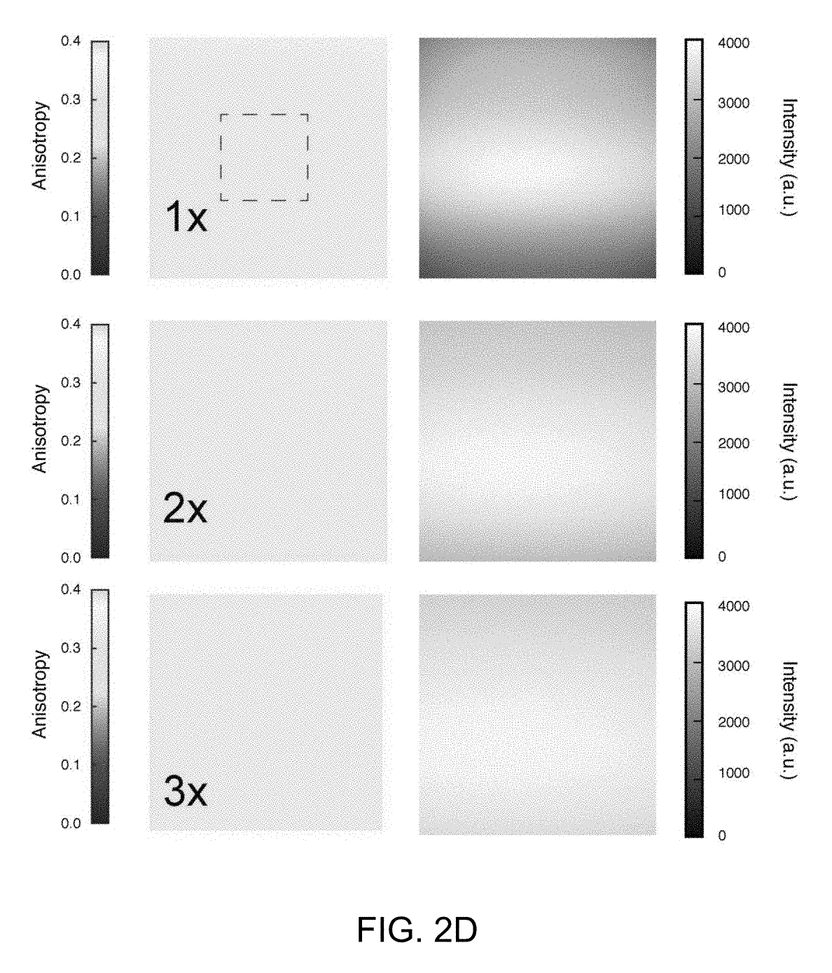

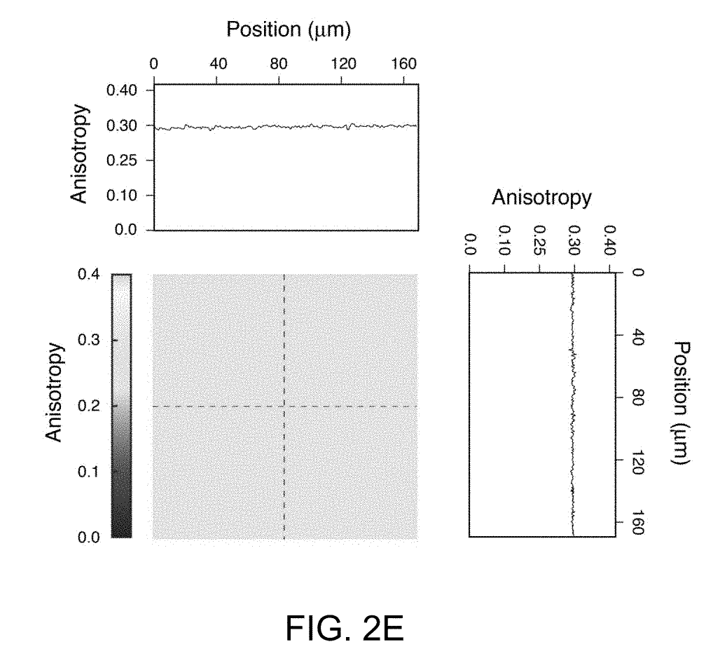

The imaging system of the invention was first tested by measuring the viscosity dependence of anisotropy for pentamethyl-BODIPY (Me5-BODIPY), an ideal fluorophore for FA (Supplementary Information: Fluorescence lifetimes), in increasing concentration of aqueous glycerol, as illustrated in FIGS. 2A and 2B. FIG. 2A shows results obtained from two photon images of sample drops of Me5-BODIPY (with varying concentrations, 0% . . . 95%, of glycerol, sandwiched between two microscope cover slips) and calculating the anisotropy of each pixel. Average±stdev (n=6), fitted curve was added for trend visualization. As shown in FIG. 2B, in pure aqueous solution (I) Me5-BODIPY is free to rapidly rotate on a timescale shorter then its fluorescence lifetime τ. This implies that after two-photon absorption, Me5-BODIPY molecules will emit photons along a direction in space uncorrelated with the one of the exciting photons (isotropic emission). Therefore the fluorescence signal will distribute equally among the two linearly polarized orthogonal state of polarization detection channels, with the two images presenting very similar values of intensity. At high values of viscosity (II) the rotational correlation time τ_{θ} is longer than the fluorescence lifetime τ. The emitted photon will therefore maintain a strict correlation with the polarization of the excitation beam with one channel brighter then the other (anisotropic emission). As shown, the measured anisotropy increased with increasing viscosity.

The superior photoselectivity by two-photon excitation compared to single photon absorption significantly increased anisotropy values, through enhanced photoselection, resulting in increased sensitivity, as illustrated in FIG. 2C. In FIG. 2C, panel (A) shows Me5-BODIPY anisotropy dependence on viscosity as measured by MFAM. Data are an average±stdev (n=6). Panel (B) shows single photon (SP) plate reader measurements of the same samples as in panel (A). Data are an average±stdev (n=3). Panel (C) shows biotin-BODIPY binding to NeutrAvidin as measured with MFAM, with (open symbols) or without (filled symbols) the presence of 10× unlabeled free biotin as competitor; average±stdev (n=3), curve fit (black lines) added for trend visualization. Panel (D) shows Biotin-BODIPY binding to NeutrAvidin as measured with single photon plate reader, with (open symbols) or without (filled symbols) the presence of 10× unlabeled free biotin as competitor; average±stdev (n=3), curve fit (black lines) added for trend visualization. Although high numerical aperture objectives are well known to produce distorted anisotropy values at the periphery of an image (with small impact on-axis), restricting the field of view eliminates these aberrations, as illustrated in FIGS. 2D and 2E. In FIG. 2D, anisotropy images of a fluorescent microscope slide are provided with varying sizes of field-of-view (1×: 600×600 microns, 2×:300×300 microns, 3×: 160×160 microns). The field-of-view is selected by restricting the scanning area while keeping constant the number of pixels within the images and the integration time per pixel (digital zooming). Within the 1× field-of-view, edge artifacts are present while galvo scanning the image. At 3× digital zooming, the anisotropy is constant over the entire field-of-view. In FIG. 2E, an anisotropy image of a fluorescent microscope slide is provided over a field of view of 160×160 squared microns. Within the field of view no edge artifacts due to the high numerical aperture in the objective are present and the anisotropy is constant. Top and right, anisotropy profiles along the orthogonal dashed lines centered across the image.

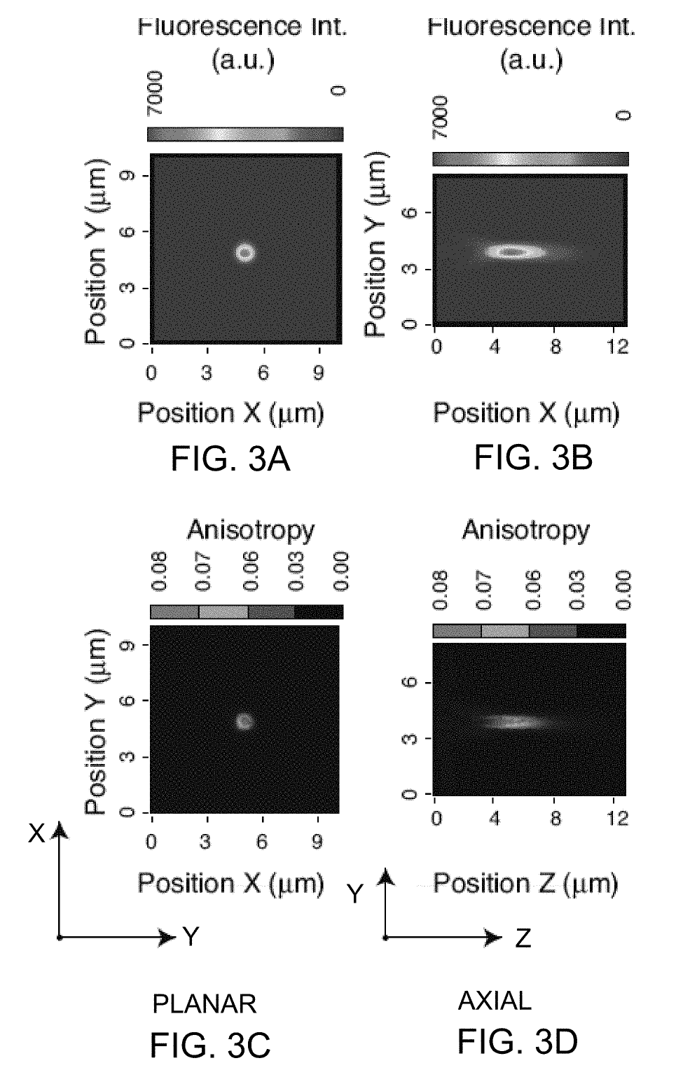

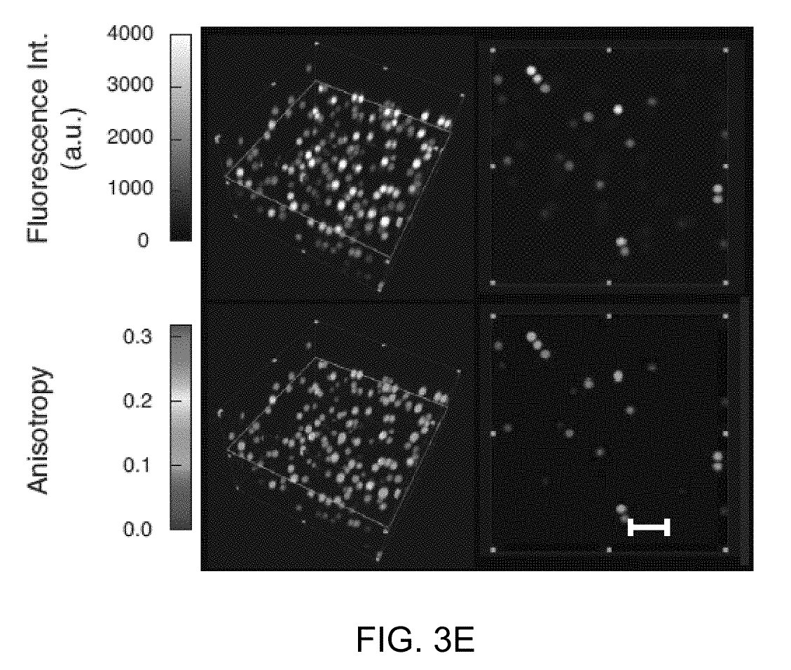

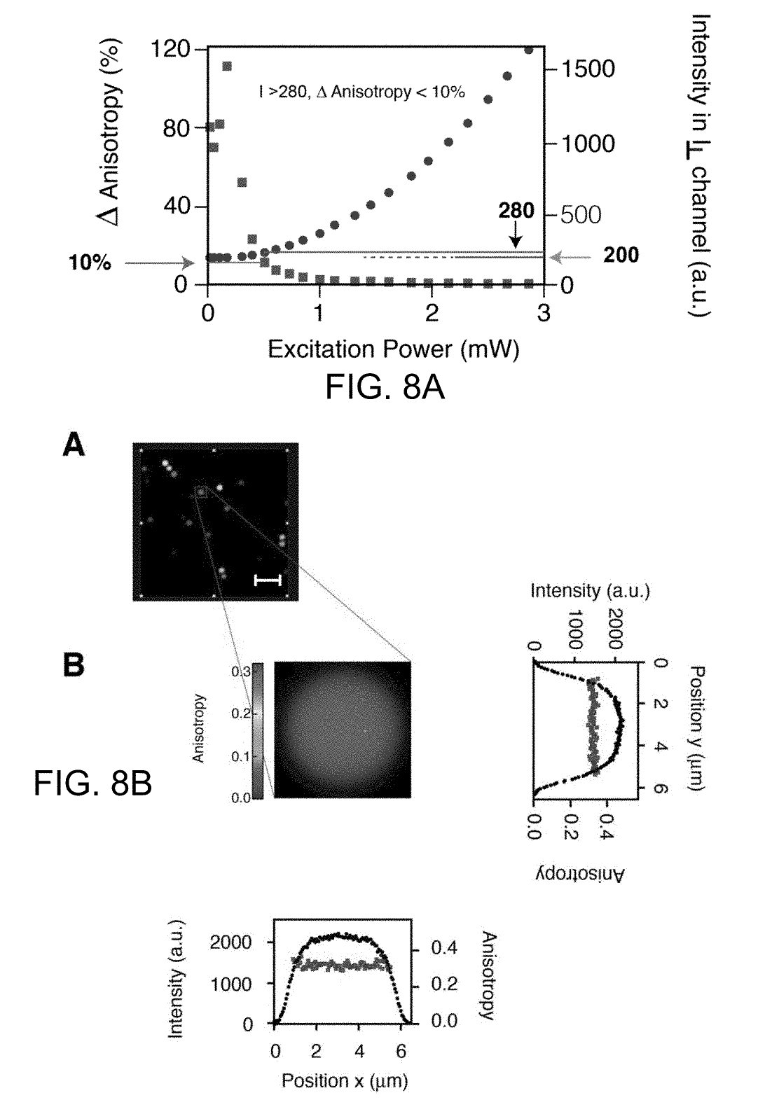

The resolution of the imaging system was determined using fluorescent microspheres. Both planar and axial measurements of a microsphere point spread function (FIGS. 3A, 3B, 3C, 3D) demonstrate the high optical resolution of FA, making MFAM ideal for 3D intracellular imaging In FIGS. 3A through 3D, the images show planar and axial microscope fluorescence anisotropy and plain fluorescence images of a fluorescent microsphere. 2D reconstructions of a mixture of two fluorescent microspheres populations with high and low anisotropy (see also FIG. 8B, 9A) demonstrate that MFAM can well separate the two populations. Anisotropy images color-coded based on anisotropy values. Right: planar images across the transversal mid lines (box). Top: fluorescence. Bottom: anisotropy. Scale bar: 20 microns. The calculated anisotropy error in each pixel increases at the edges of the microspheres, a consequence of low count rates, resulting in some noise artifacts and loss of anisotropy (FIGS. 8A, 8B). However, anisotropy remained constant above a threshold that is determined by acquisition parameters and intrinsic noise (FIG. 8A).

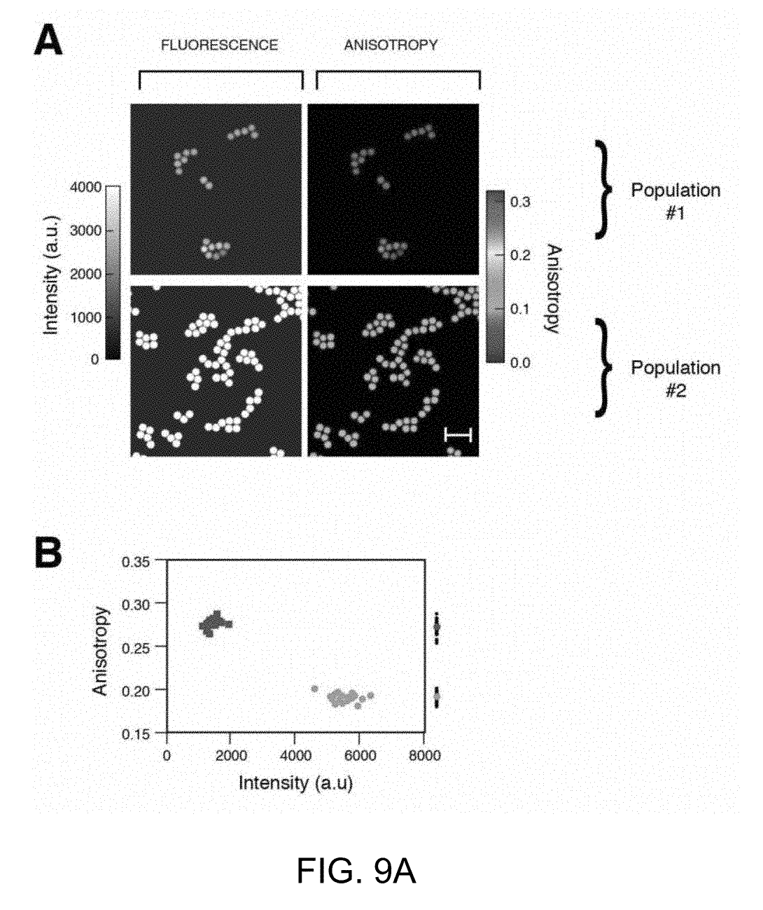

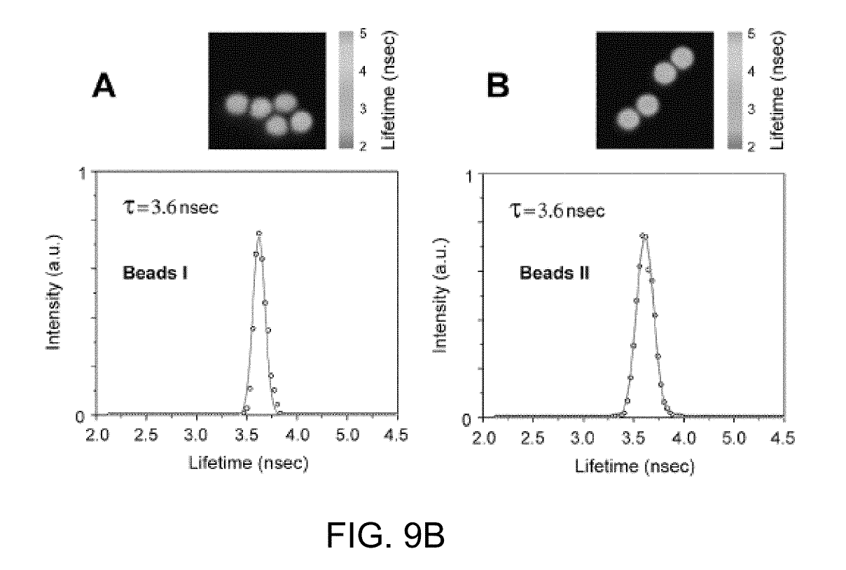

The excellent optical sectioning properties of the embodiment of the invention to carry out tomographic MFAM imaging of an optical phantom simulating a bound/unbound 3D environment. To this end, two highly homogeneous populations of green fluorescent microspheres with distinct anisotropy values (FIGS. 9A, 9B) were suspended in a 2% agarose solution (FIG. 3E). In both the 3D FA colorcoded reconstructions and the optically sectioned planes, the two populations of microspheres are distinguishable throughout the entire phantom depth (ca. 90 microns) and assigned the correct anisotropy-based color (FIG. 3E and FIG. 8B, part B).

### Imaging Drug-Target Engagement in Cells.

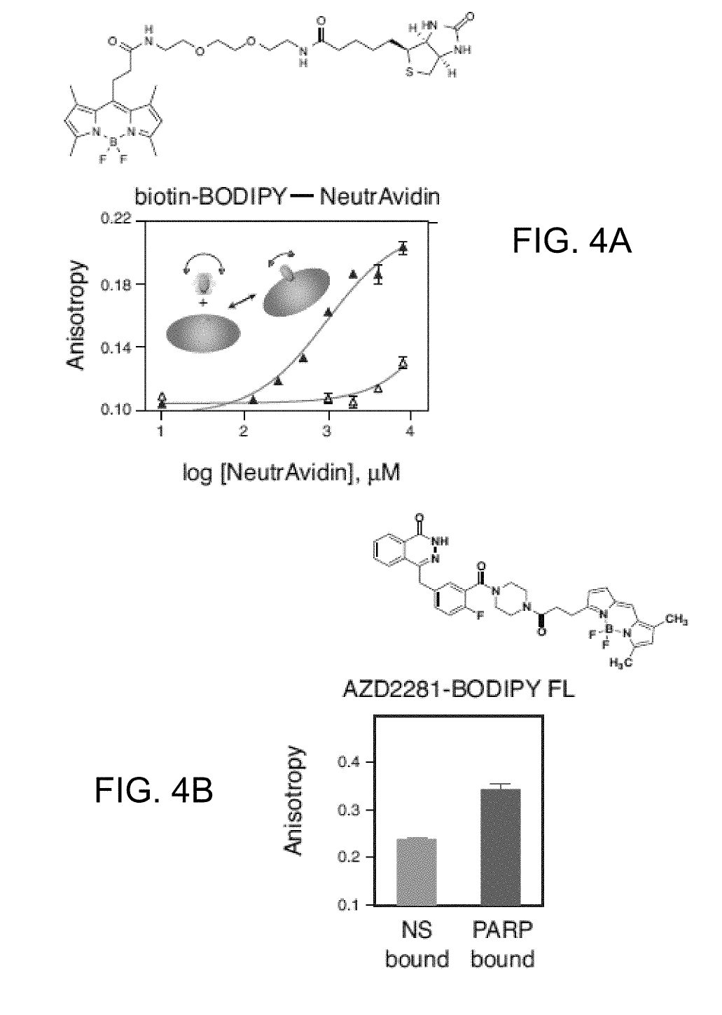

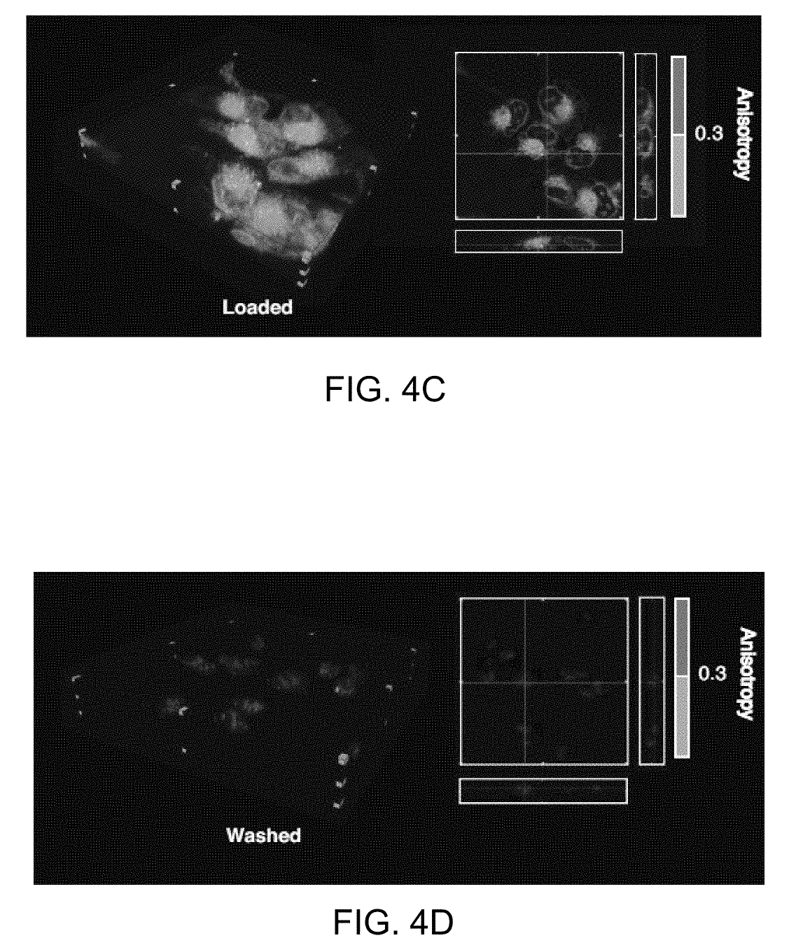

FIGS. 4A, 4B, 4C, and 4D illustrate the results of imaging of the live-cell-to-target engagement. FA has traditionally been used to measure binding of small fluorescent molecules to a larger target biomolecule. When bound, the increased molecular mass of the probe-target complex will result in a higher rotation correlation time τθ limiting molecule rotation and increasing FA (FIG. 4A), while a shift in fluorescence lifetime could also change FA. FIG. 4A shows the average±stdev (n=3); curve fits added for trend visualization. Inset illustration: comparison between the rotation of a free fluorophore in solution and a fluorophore bound to a protein. Due to the large difference in size of the ligand and the receptor, the increase in fluorescence anisotropy following binding is large. Depending on its state (bound/unbound), a single fluorescent molecule can produce two values of anisotropy, and, because anisotropy is an additive property, the measured pixel value in an FA image is the fraction-weighted sum of the two possible anisotropy values within a voxel. MFAM fluorescence anisotropy measurements of Me5-BODIPY labeled Biotin (Biotin-BODIPY) indeed show an increase in anisotropy as a function of binding to NeutrAvidin (FIG. 4A) with a similar trend to single photon measurements (see FIG. 2C), due to a change in τθ.

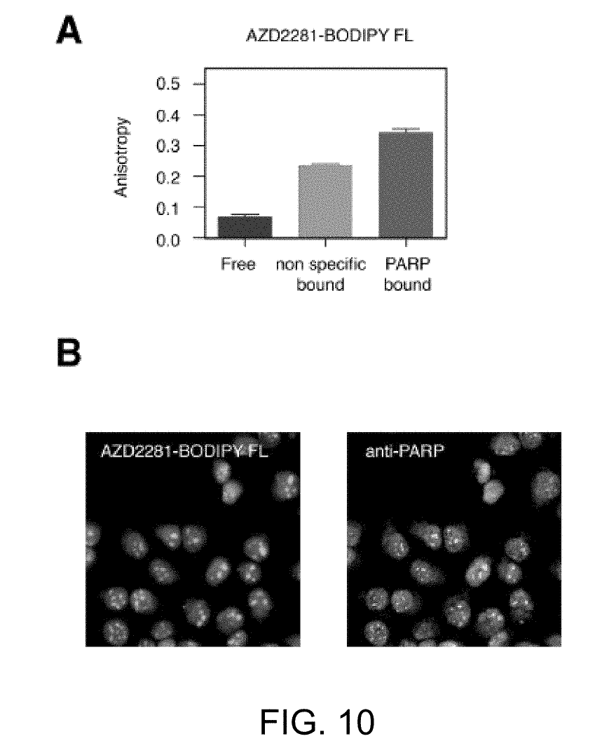

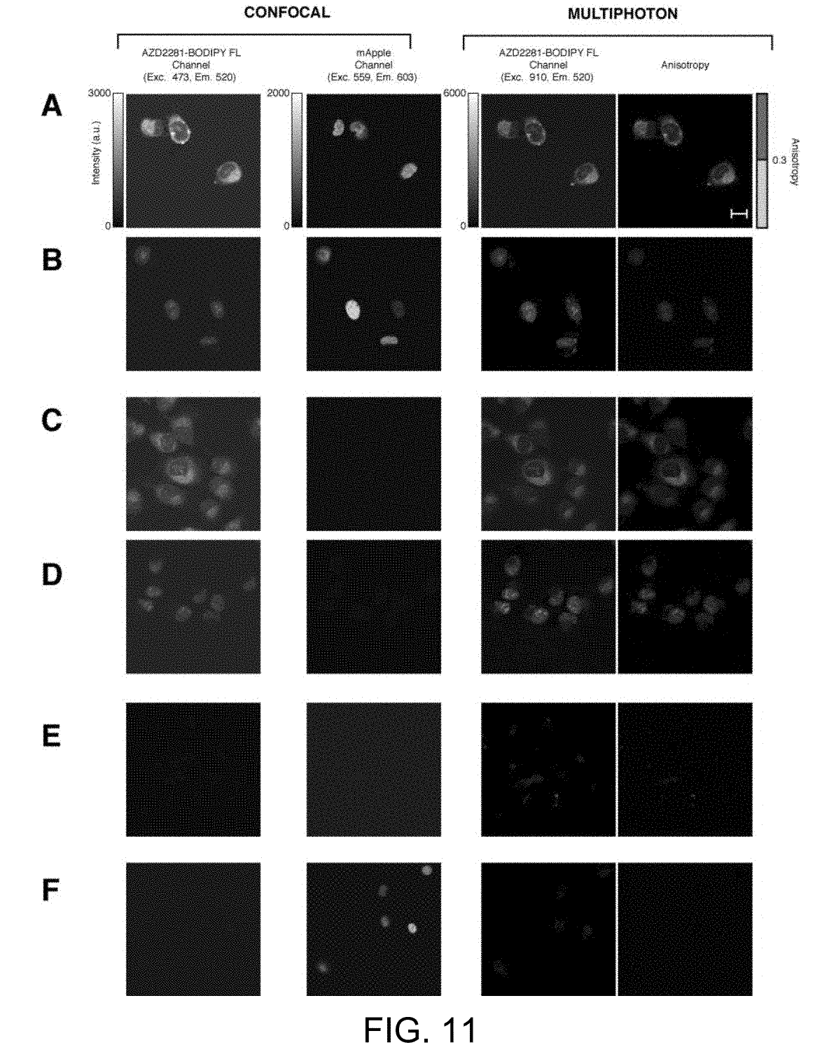

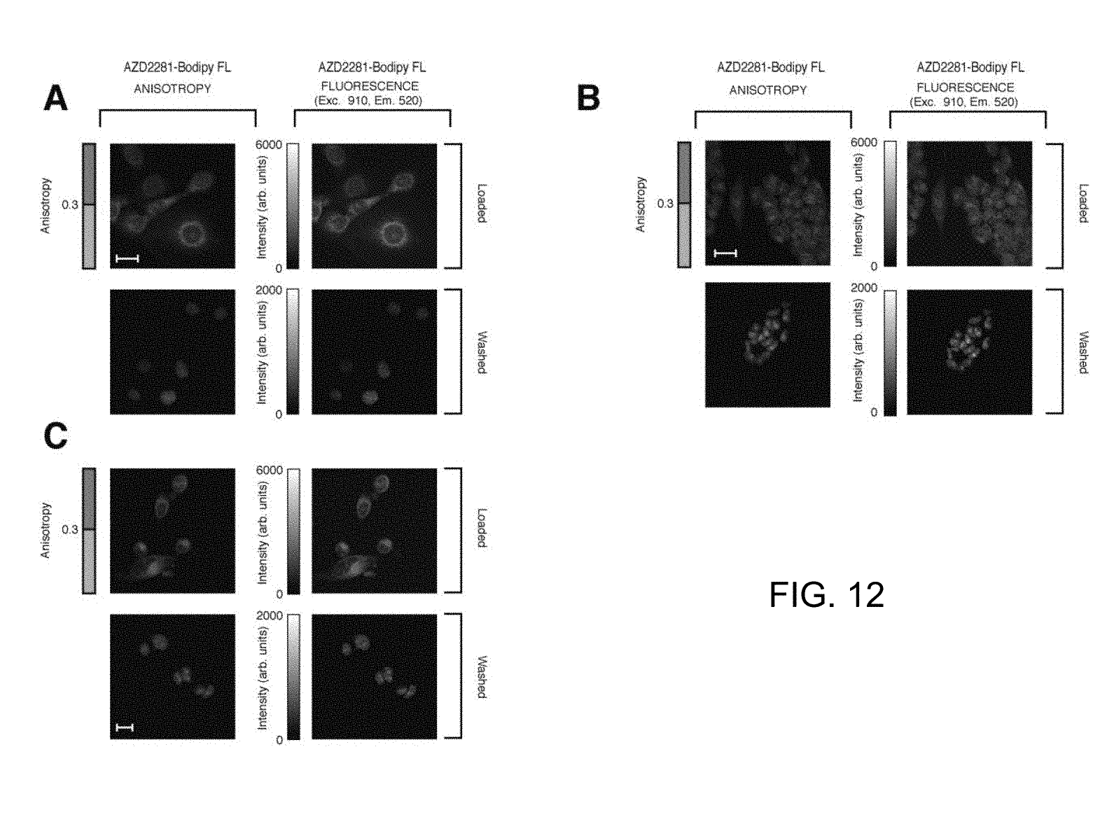

While dyes presenting longer lifetimes could be considered as alternative candidates, BODIPY was chosen due to unique characteristics that allow intracellular imaging Specifically: i) BODIPY is relatively non-polar with the chromophore presenting electrical neutrality, therefore minimizing perturbation to the modified drug; ii) the relatively long lifetime (the BODIPY we use here has a measured lifetime ˜4.0 nsec) makes it particularly suitable for fluorescence polarization-based assay; iii) BODIPY is highly permeant to live cells, easily passing through the plasma membrane, where it accumulates over time; iv) it has a high extinction coefficient (EC>80,000 cm−1M−1) and a high fluorescence quantum yield (often approaching 1.0, even in water); v) it presents a lack of ionic charge and spectra that are relatively insensitive to solvent polarity and pH; and, vi) finally, it has a large two photon cross section. Although most BODIPY dyes enjoy a relatively long lifetime, dyes such as Cy3 and the Alexa dyes will be inefficient for fluorescence anisotropy imaging, with their lifetimes so short that the anisotropy of the unbound probe will be near the fundamental anisotropy, and hence indistinguishable from the bound probe. Conversely, fluorophores with extremely long lifetimes, or phosphorescence emission, are also unsuitable as the increase in rotation correlation time will not be large enough to increase the anisotropy. It is therefore important to characterize the lifetime, by fluorescence lifetime imaging microscopy (FLIM), of the possible candidate dyes for drug labeling that could be potentially used for two photon fluorescence polarization imaging. Also, dyes presenting changes in their quantum yield upon binding will bias the readout value of total anisotropy affecting the measured binding isotherm. To test the MFAM imaging approach in a relevant drug-target system, we chose to target poly(ADP-ribose) polymerase (PARP) with the small molecule inhibitor Olaparib (AZD2281) which had been modified to bear a BODIPY-FL handle. This model system and its cellular location had previously been well validated. PARP comprises a family of enzymes that are required for DNA repair, and therefore present a potential chemotherapeutic target through inhibition. Due to the high molecular weight of PARP1 (˜120 kDa) a significant increase in anisotropy is observed for “target-bound” over “free” or “intracellular drug” AZD2281-BODIPY FL, respectively (FIG. 4B and FIG. 10A). An anisotropy threshold can then be assigned to distinguish between the bound states and MFAM intracellular imaging of drug target engagement can be obtained in 3D (FIGS. 4C, 4D: dark color, PARP bound; light color, “intracellular drug”). When incubated with AZD2281-BODIPY FL, we observed rapid accumulation throughout the entirety of each HT1080 cell. Intracellular drug was present in the cytoplasmic region, while bound drug was present in the nucleus (FIG. 4C and FIG. 11), which co-localized with PARP immunostaining (FIG. 10B). Following extended washing cycles, the cytoplasmic AZD2281-BODIPY FL is cleared, while the nuclear, bound drug remains (FIG. 4D) Similar nuclear binding of AZD2281-BODIPY FL was observed in other cell lines reported to express PARP as well (FIG. 12).

Dyes other than BODIPY can be also used to fluorescently label a molecule or ligand, and BODIPY was here chosen as a possible examples of fluorophore due to its desirable characteristics.

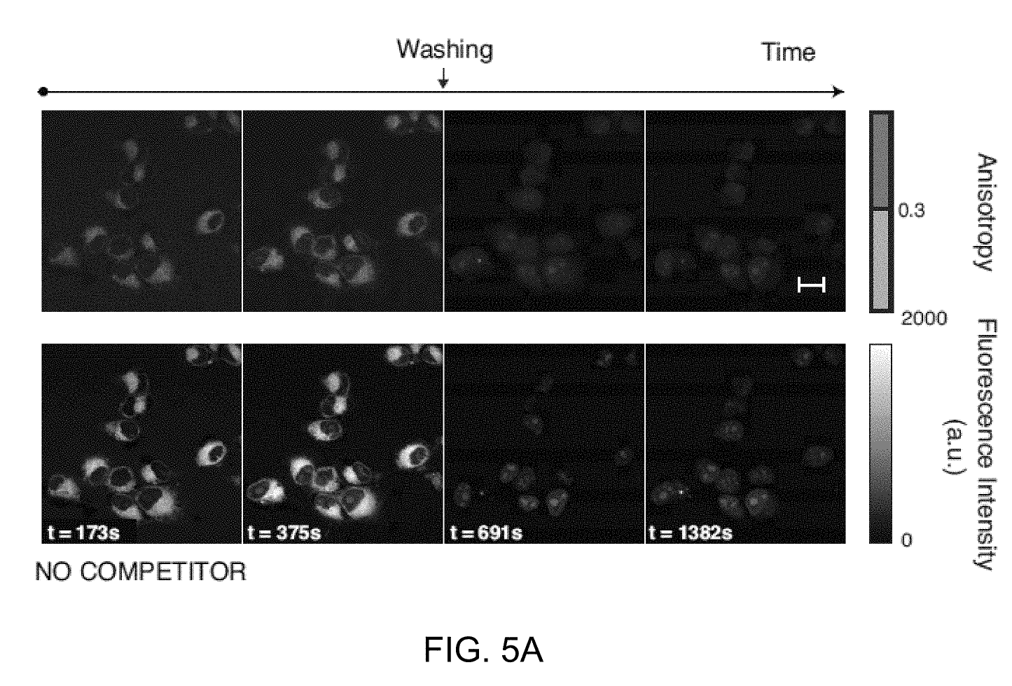

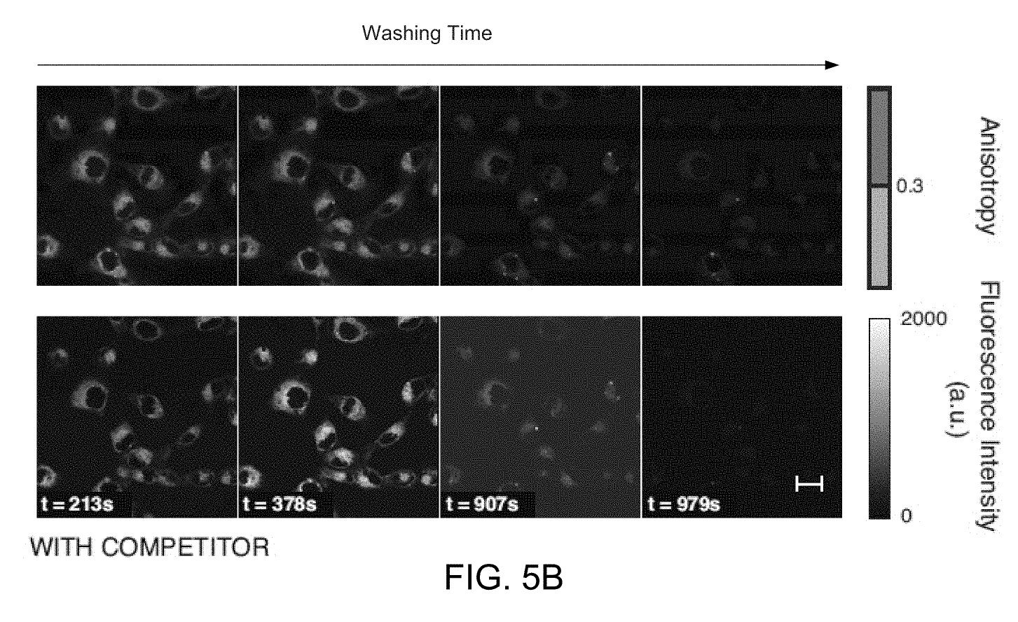

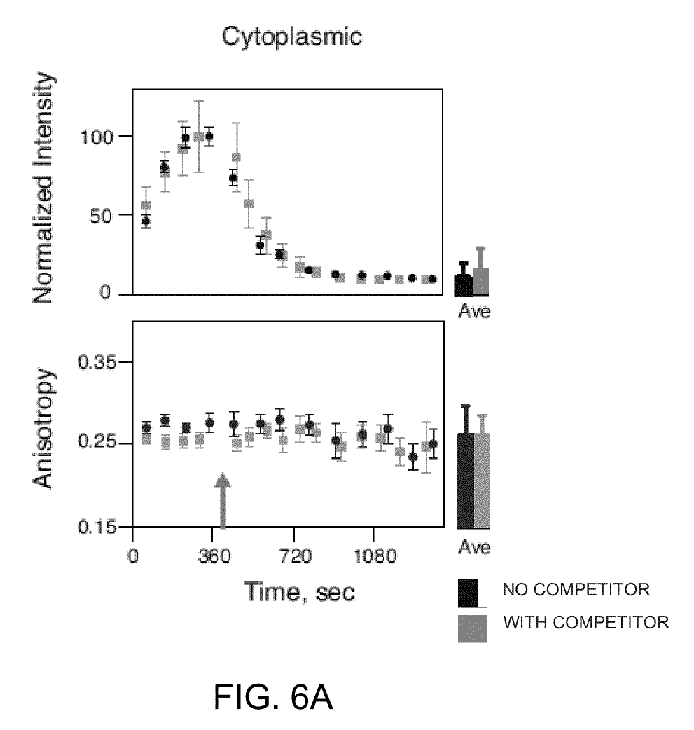

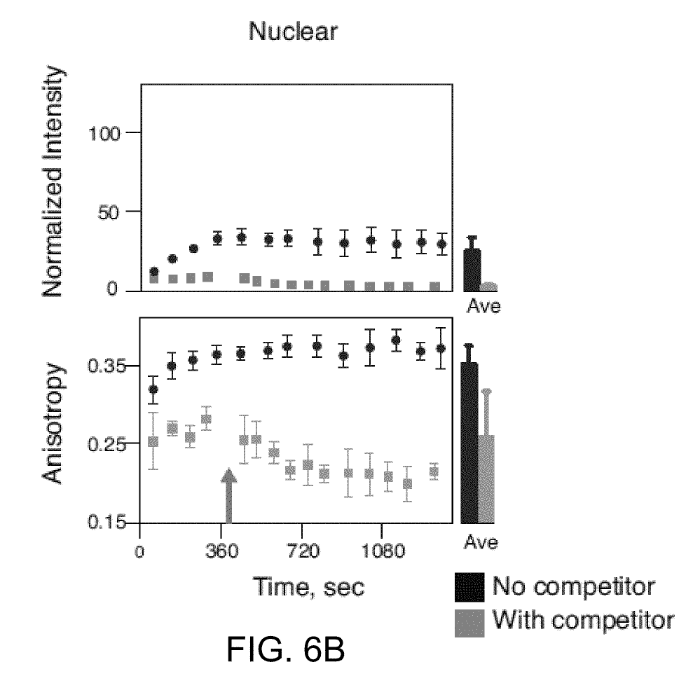

In reference to FIGS. 5A and 5B, the results of the real time in vitro measurements show that AZD2281-BODIPY FL accumulated in the cytoplasm significantly more than in the nucleus, which is likely the result of interactions with intracellular membranes. Yet, only the nucleus presents high values of anisotropy, suggesting PARP binding (FIG. 5A). The high nuclear anisotropy is not observed in the presence of unlabeled AZD2281 as competitor (5×) (FIG. 5B), which further suggests the high anisotropy measured in the nuclei was due to drug target binding and not induced by potential artifacts, such as viscosity. In addition, there was no target binding of AZD2281-BODIPY FL in the cytoplasm, as demonstrated by the significant difference between nuclear and cytoplasmic anisotropy throughout the course of loading and washing as well as the insignificant difference between cytoplasmic anisotropy in the non-competitive and competitive experiments (as shown in FIGS. 6A, 6B). In FIGS. 6A and 6B, the graphs show normalized intensity and anisotropy as a function of time for HT1080 cells loaded with AZD2281-BODIPY FL and washed. Values are measured in both the cytoplasmic (FIG. 6A) and nuclear (FIG. 6B) regions of the cells in the absence (black circles) and presence (gray squares) of 5 fold higher concentration of unlabeled AZD2281 (competition). Points in the graphs refer to a single experiment, average±stdev (n=6 cells). Also shown at the right of each figure, average±stdev at the end of the wash in the absence (black bars, n=42 cells, 7 separate experiments) and presence (gray bars, n=36 cells, 6 separate experiments) of unlabeled AZD2281 (5×). Bars are representative of 7 and 6 different experiments, respectively. Arrows indicate switch from loading to washing. Fluorescence intensity refers to the sum of both perpendicular and parallel channels.

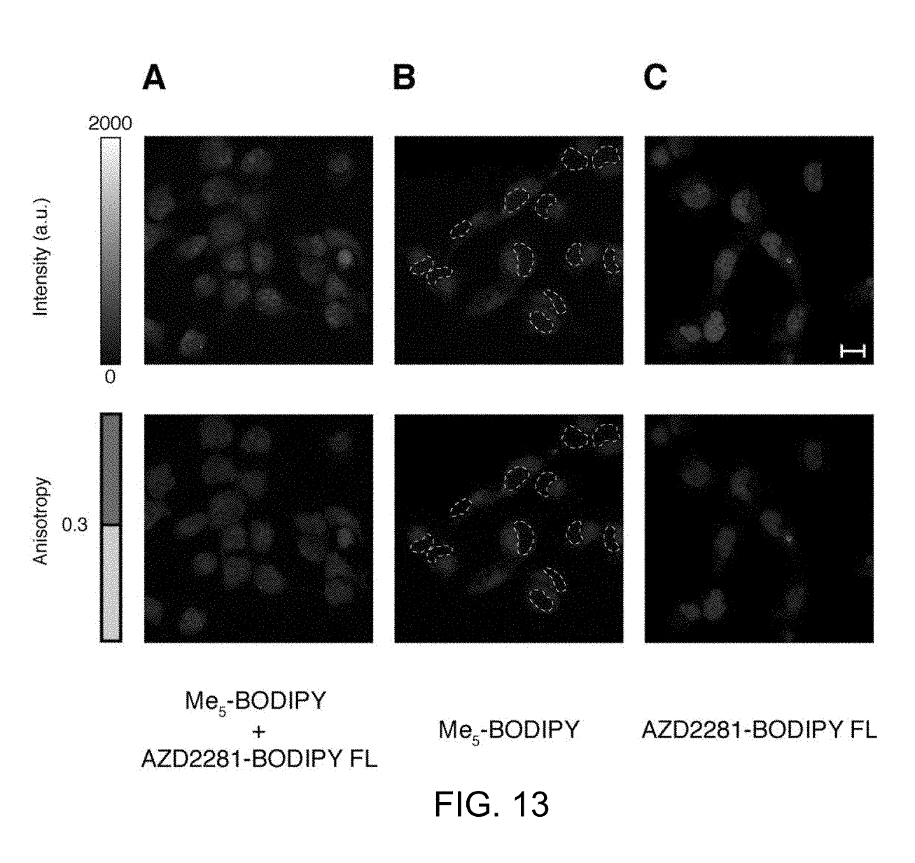

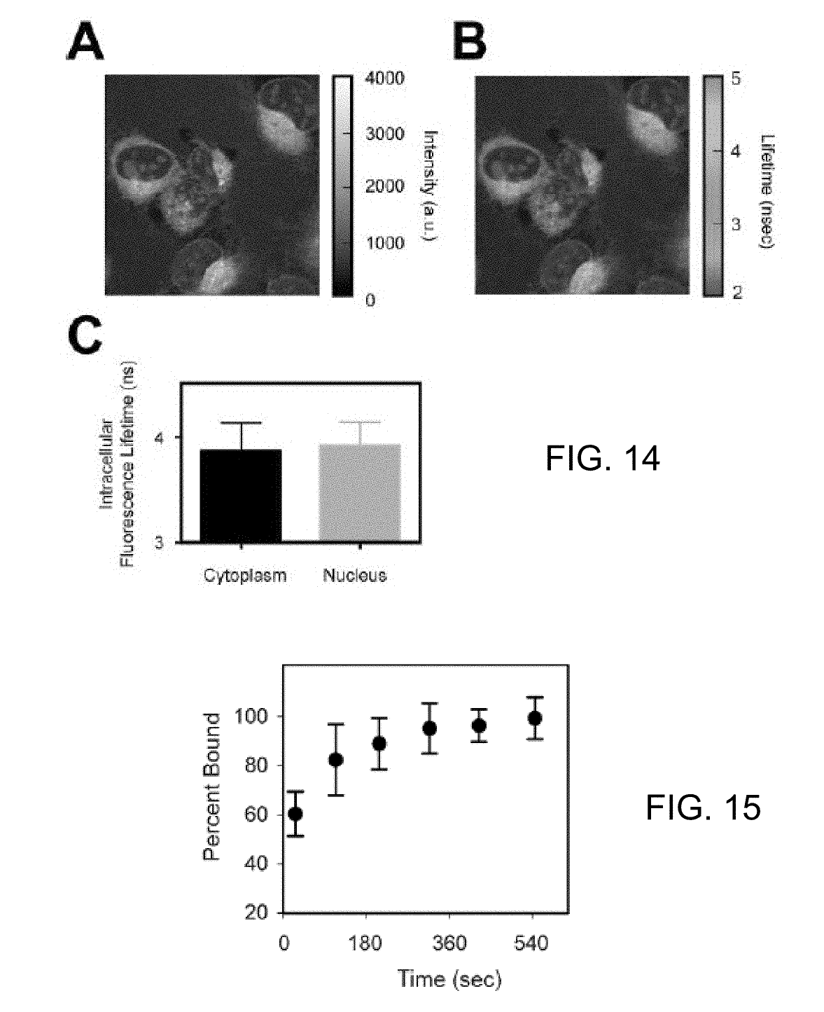

Constant anisotropy with decreasing intensity in the cytoplasm in both non-competition and competition experiments indicates that homoFRET was not the cause of the lower anisotropy (FIGS. 6A, 6B). Additionally, high nuclear anisotropy is not caused by the BODIPY FL itself (FIG. 13). Finally, there was no significant difference in fluorescence lifetime between nuclear and cytoplasmic regions in loaded HT1080 cells (FIG. 14). Through washing and competition experiments, bound and unbound values of anisotropy in the nucleus can be determined, and the percentage of target bound AZD2281-BODIPY FL can be calculated at any point in time (FIG. 15).

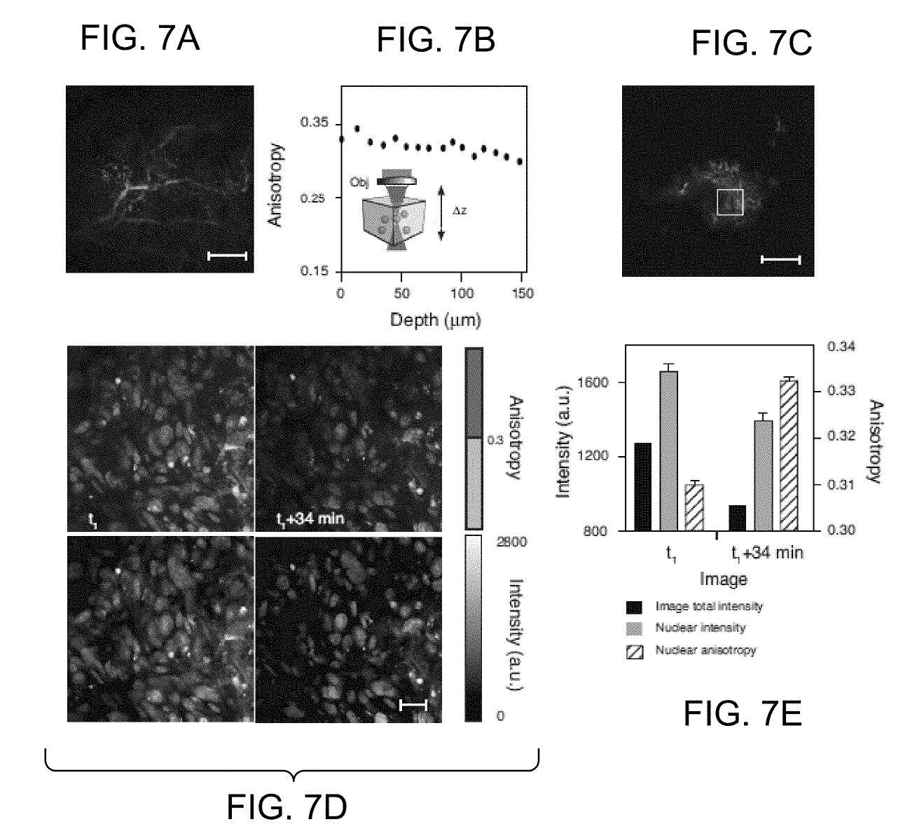

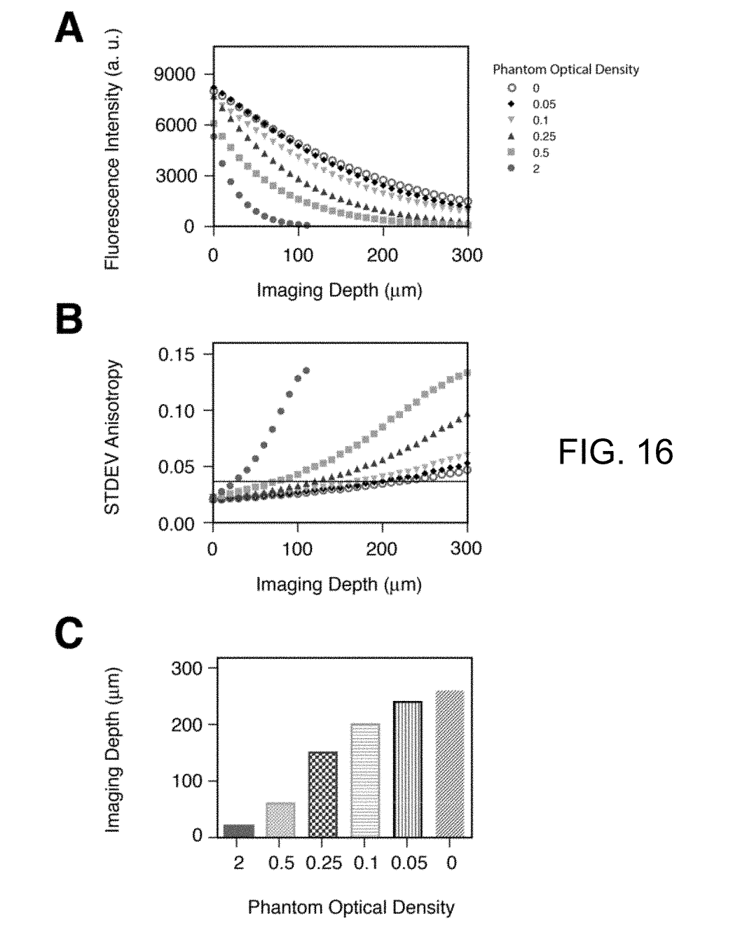

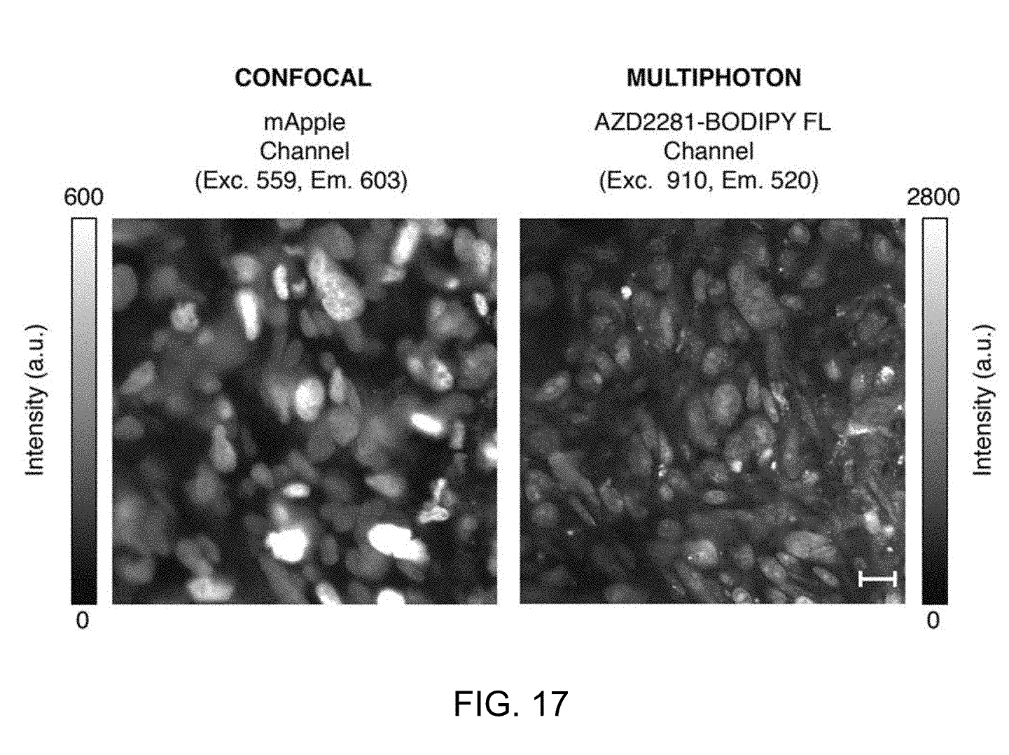

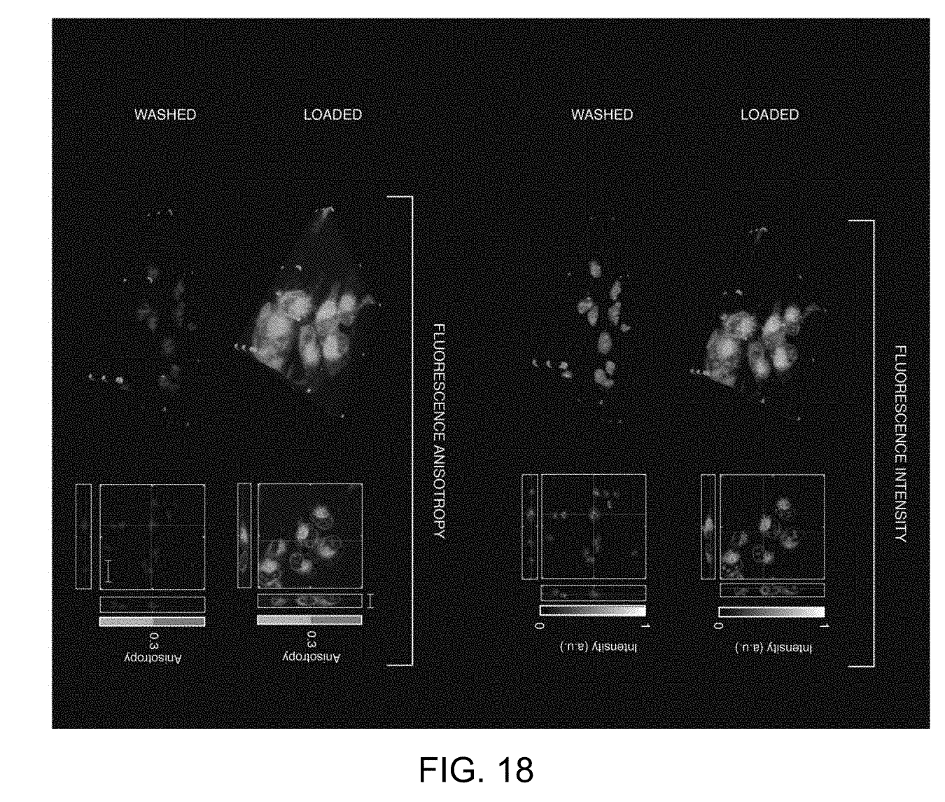

The MFAM system of the invention was also used for in vivo imaging In biological diffusive samples multiple scattering events limit the imaging depth by reducing the number of excitation photons in the focal area while decreasing the number of collected photons. A decrease of the degree of polarization with resulting lower values of anisotropy is therefore present as evidenced on tissue phantom measurements (FIG. 16). To better characterize how diffusion and absorption limit the effective anisotropy imaging depth we first injected fluorescent microspheres into superficial tissue within a nude mouse dorsal window chamber (FIG. 7A). In vivo MFAM measurements indicated a slight depth-dependent loss of anisotropy (FIG. 7B), with a 10% loss at 100 microns, which, based on the anisotropy difference in binding measurements, does not affect target engagement measurements.

After determining that our technique is viable in an in vivo setting we measured drug target engagement in a mouse in vivo. Intravenous delivery to an implanted HT1080 cell tumor showed AZD2281-BODIPY FL diffusion into the cancer cells (FIG. 7C). Cells expressing nuclear mApple-labeled H2B, which did not effect AZD2281 anisotropy measurements (FIG. 11), were used to locate the tumor. Binding of AZD2281-BODIPY FL to PARP in the nucleus occurred immediately upon drug infusion (FIG. 7D). The bound fraction of the drug was retained in the nucleus while the unbound extracellular and cytoplasmic drug was cleared away over time (FIG. 7D). Both the nuclear and overall fluorescence intensity decreased over time, however the nuclear anisotropy increased as unbound AZD BODIPY FL was cleared (FIG. 7E).

As a skilled artisan would readily recognize, the ability to measure the pharmacology of drugs on a molecular level in live cells represents one of the greatest challenges in chemical biology and drug discovery and remains a not-addressed need because to-date, there are no demonstrated methods for direct measurements. Subsequently, all information is based on indirect or artificial approaches that do not provide the spatiotemporal resolution and accuracy required to establish reliable models and/or do not occur in biologically relevant settings.

The present invention provides a response to such long-felt need. The present application discloses a promising novel approach (referred to as MFAM) utilizing the multiphoton fluorescence anisotropy microscopy system which, for the first time, allows direct visualization of target bound versus unbound small molecule drugs in real time. Using a chemotherapeutic compound, the proposed approach was proved to be not only applicable to live cultured cells but also enabling with respect to the real-time imaging of drug target engagement in vivo and with submicron resolution. The disclosed technique does not require separation between bound and free compound, is not limited to equilibrium analysis and does not affect the biological settings. As such, MFAM offers a new and fundamental imaging platform for accelerating translational drug development through insight into in vivo drug activity and inefficacy.

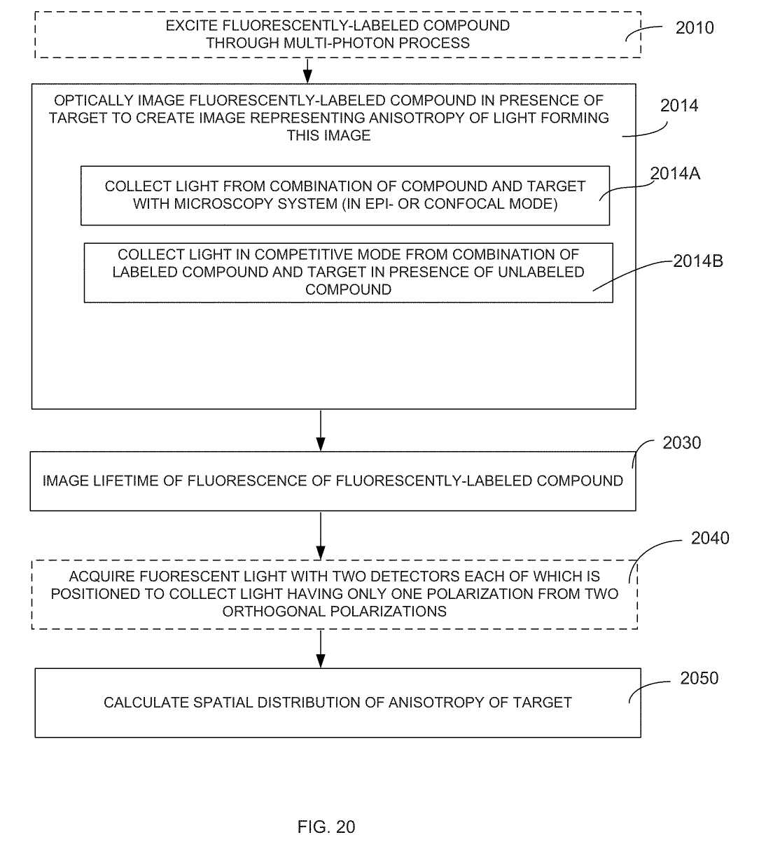

FIG. 20 provides a flow-chart illustrating some steps of a method of the invention. Optically excited (at step 2010) fluorescently-labeled compound (a drug molecule, in one implementation) is introduced to a target (such as a living cell) and is optically imaged, at step 2014, to form an image representing anisotropy of light emanating from the target-compound combination. The process of optical imaging includes collection of light with a microscopy system, 2014A, and/or collection of light in a competitive mode when an unlabeled compound is also present, 2014B. As part of the optical imaging, imaging of lifetime of fluorescence of the fluorescently labeled compound is performed at step 2030. Acquisition of light is optionally performed with two detectors through an optical system configured such that each of the detectors acquires light having only one state of polarization from two different states of polarization, 2040. Calculation of spatial distribution of anisotropy of imaged target is performed at step 2050.

### APPENDIX

Cell Culture

HT1080 cells (ATCC) stably expressing H2B mApple fluorescent protein were cultured in DMEM with 10% FBS, 1% pen-strep and 100 μg/ml geneticin (Invitrogen). HT1080 cells were cultured in DMEM with 10% FBS and 1% pen-strep. MDA-MB-436, HCC1937, and MHH-ES1 cells were cultured in RPMI with 10% FBS and 1% pen-strep. Cells were plated onto 25 mm #1 cover glass for in vitro imaging.

Tumor Model

All animal experiments were performed in accordance with the Institutional Animal Care and Use Committee at Massachusetts General Hospital. Female 20-weeks old nude mice (Cox-7, Massachusetts General Hospital) were used. All surgical procedures were conducted under sterile conditions and facilitated through the use of a zoom stereomicroscope (Olympus SZ61). During all surgical procedures and imaging experiments mice were anesthetized by isofluorane vaporization (Harvard Apparatus) at a flow rate of 2 L/minute isofluorane: 2 L/minute oxygen. The body temperature of the mice was kept constant at 37° C. during all imaging experiments and surgical procedures. Dorsal skinfold window chambers (DSC) were implanted one day prior to imaging following a well-established protocol. Briefly, the two layers of skin on the back of the mouse were stretched and kept in place by the DSC. One skin layer was surgically removed and replaced by a 12-mm diameter glass cover slip positioned on one side of the DSC allowing for convenient access and imaging of the tumor area. A spacer located on the DSC prevented excessive compression of both tissue and vessel guaranteeing good vascularperfusion within the tumor region.

HT1080 H2B mApple cells were harvested by trypsinization (0.25% trypsin:EDTA) and resuspended in PBS. Mice were anesthetized and approximately 106 cells (100_1 1×PBS) were injected subcutaneously into the back of female Nu/Nu mice (Cox-7, Massachusetts General Hospital, Boston, Mass.) aged 20-25 weeks in a 1:1 mixture of Matrigel (BD Biosciences). Cells were injected using a 0.5-mL insulin syringe with the needle bent at 90 degrees to better control the position of the injection site. In order to allow for the tumor to be established and neovascularization to occur, the tumors were allowed to grow for 1-2 weeks before DSC implantation.

Optical Characterization of the System

All polarizer, optical filters, polarization beamsplitter, half-wave plate, and Glan-Thompson polarizer were tested and characterized. Light from the laser was first linearly polarized using a Glan-Thompson polarizer and then aligned along a defined arbitrary axis with the use of a half waveplate. Light at the entry of the objective was measured using a polarizer and a photodetector to confirm the state of polarization remained linear along its path to the objective. Photodetectors were tested for any polarization dependence. The path from the objective to the photodetectors was also tested to assure that equal distribution of power is present between the two detectors. Voltage of the two photodiodes was slightly adjusted in order to fine tune equal signal detection. The noise contribution of the two detectors was equal for all in vitro and in vivo measurement conditions. The two detectors responded with the same linear curve along the measurement range.

Calibration of the multiphoton fluorescence anisotropy microscopy systems was performed using a set of angle-adjustable linear polarizer placed in front of the detectors, and at the entry of the objective. Fluorescein in water at room temperature was used to fine tune the voltage gains on the two individual PMT sensors. 5 μl of solution were placed between a microscope slide and a cover glass and imaged. Settings were regulated such that 2 μM fluorescein solution produced an anisotropy of 0.004 after correction of the G factor. The gains settings were then maintained throughout the entirety of all measurements.

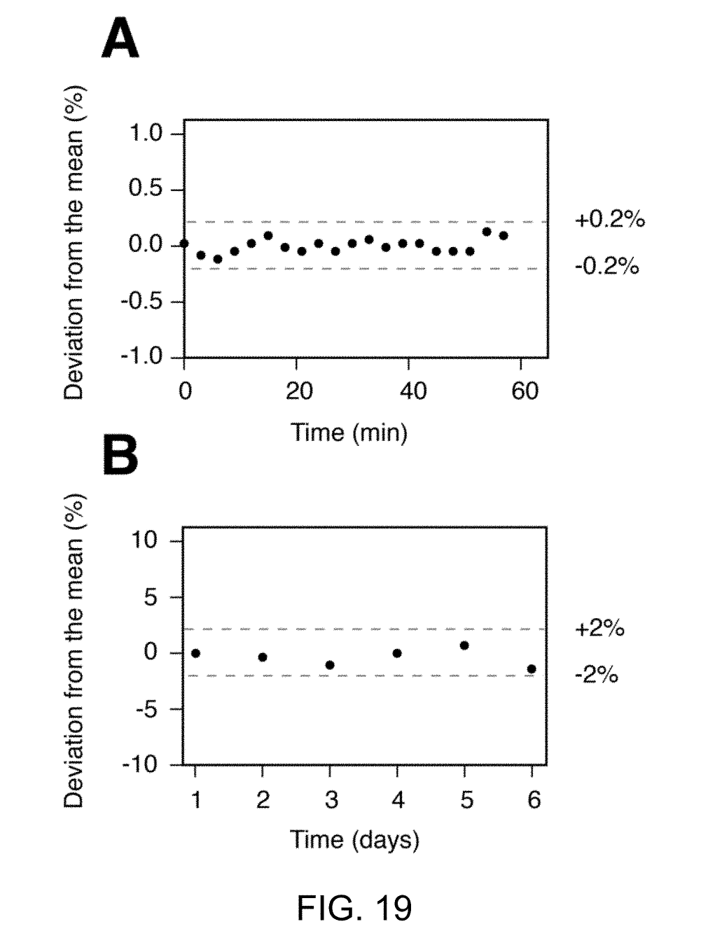

To check reproducibility over days, fluorescence slides containing uniformly distributed fluorophores were measured before each imaging sessions. Images of three different slides (each one with a different fluorophore) were taken during each imaging session to confirm that the measured anisotropy during the session matched the previous measurements. Images of the slides were taken over various time periods and at varying excitation intensity for system characterization. Thermal variation can cause slight difference on a day-to-day basis. To compensate for them the microscope is located within a thermally stable isolating cage, mounted on an aluminum frame. Measurements over time within the same day and over several days indicate strong reproducibility in FA measurements (FIG. 19). Polarization distortions due to dichroic beamsplitter reflections and the objective's high numerical aperture, such is the requirement for multiphoton microscopy, can lead to anisotropy artifacts in particular when imaging over the entire objective field of view. While compensation could be used through different calibration methods, images collected over a restricted field of view eliminate any edge artifact (FIGS. 2D, 2E; see also Appendix, below: Loss of polarization through imaging).

Me5-BODIPY was brought up in DMSO (Sigma) to a 1 mM stock solution. Solutions of a final concentration of 20 μM Me5-BODIPY in DMSO were mixed with glycerol (Sigma) to create varying concentrations of glycerol. Images of 5 μl drops of solution inserted between the cover glass were taken at each glycerol concentration in triplicate.

3D Anisotropy Phantom

Six microns green-fluorescent microspheres (InSpeck Microscope Image Intensity Calibration Kits, Invitrogen) were used for demonstrating optical sectioning capabilities. Each kit consists of seven different types of microspheres with fluorescence intensities ranging from very low to very bright (100%, 30%, 10%, 3%, 1%, 0.3%, and non-fluorescent). The fluorescence intensity of the microspheres within each vial is defined with respect to that of the microspheres with the highest fluorescence (i.e. 100%). We selected one vial containing the brightest microspheres (i.e. 100%) and another vial containing the next brightest (30%) microspheres. The fluorescence intensity of the microspheres in each vial is highly homogeneous as shown in FIG. 9A. Importantly, their value of anisotropy is not dictated by the lifetime (see FIG. 9B) or mobility of dye within the microspheres, but instead by a concentration-dependent effect (homo-FRET).

Due to homo-FRET, the two populations of microspheres present different values of anisotropy with a highly homogenous distribution (0.274+/−0.008 and 0.193+/−0.005; see FIG. 9A). The microspheres are therefore useful for testing anisotropy distributions in phantoms. The two populations of microspheres were mixed in equal proportion, suspended in 2% agarose and allowed it to solidify between two pieces of cover glass before imaging.

Point-Spread Function Measurements.

One micron green fluorescence microspheres (Bangs Labs) on cover glass were also imaged and used for point spread function characterization.

FLIM Measurements.

Fluorescence lifetime imaging was performed using a Zeiss 710 confocal NLO laser scanning system on an upright Zeiss Examiner stand with a 40×NA 1.1 water immersion LD CApochromat objective and a Becker & Hickl TCSPC system. Two-photon excitation was achieved using a Coherent Chameleon Vision II tunable laser (680-1040 nm) that provided 140-femtosecond pulses at a 80-Mhz repetition rate with an output power of 3 W at the peak of the tuning curve (800 nm). Laser scanning was controlled by Zeiss Zen software and set to a pixel dwell time of 1.58 microseconds and 0.9-sec frame rate at 910 nm wavelength excitation.

Enhanced detection of the scattered component of the emitted (fluorescence) photons was afforded by the use of a Becker & Hickl HPM-100-40 hybrid detector, which incorporates the Hamamatsu R10467 hybrid PMT tube Imaging was performed in the dark with blackout enclosure around the microscope to exclude external sources of light during the sensitive period of FLIM measurement. Emitted fluorescence was deflected to the non-descanned light path via a 760+ mirror and emission range was limited to 500-550 nm by a Chroma filter in front of the HPM-100-40 detector. Acquisition time was typically 60 seconds with a count rate of 2-5×104 photons per second. Photon counting and electronic timing synchronization was controlled and measured with a Becker & Hickl TCSPC electronics (SPC-830) and SPCM software (Becker & Hickl GmbH) Lifetime decay of the fluorescence was analyzed with SPClmage software (Becker & Hickl GmbH).

Plate Reader Anisotropy Measurements

Single photon (SP) data were collected in a plate reader set up for fluorescence polarization measurements (Tecan Sapphire 2). A G-factor for the instrument was calculated from 2 μM fluorescein in water. Measurements were performed in 96 or 384 well plates.

Biotin-BODIPY FL and NeutrAvidin Binding

Biotin was conjugated to Me5-BODIPY (Biotin-BODIPY) and brought to 1 mM stock solution in DMSO. Biotin-BODIPY (10 μM) was mixed with varying concentrations of NeutrAvidin (Thermo Scientific) in PBS with 1% Triton X (Sigma). Each sample was imaged in triplicate as a drop between a microscope slide and cover glass. Measurements of each sample were also performed using single photon excitation in a plate reader. Measurements were also made in the presence of 100 μM free Biotin to competitively compete with the Biotin-BODIPY.

Free Molecule Anisotropy

AZD2281 labeled with BODIPY FL (AZD2281-BODIPY FL) was prepared as previously described (see Thurber, G. M. et al., Single-cell and subcellular pharmacokinetic imaging allows insight into drug action in vivo, Nat Commun, 4, 1504, (2013), for example). PARP1 (BioVision) was brought up in the manufactures recommended solution and added at 1.6× the concentration of AZD2281-BODIPY FL (5 μM) in imaging media containing 2.5% FBS. Free AZD2281-BODIPY FL (5 μM) (no PARP) in the same imaging media with 2.5% FBS and in DMSO solutions were also made. Images were taken of drops of solution between cover glass.

In Vitro Cellular Imaging

Cells on 25 mm cover glass were mounted into a closed bath perfusion chamber (Warner Instruments) and perfused with a custom perfusion system that enabled solution switching in the imaging chamber. Cells were imaged in phenol red-free DMEM with 10% FBS and 1% pen-strep. AZD2281-BODIPY FL (1 μM) was perfused into the imaging chamber followed by a washout with drug free media. Images were obtained during the entire time interval at regular time points. For competition experiments, free AZD2281 (5 μM) (Selleck Chemicals) was added to the incubating solution before during and after AZD2281-BODIPY FL addition. Me5-BODIPY was used for fluorophore control experiments.

In Vivo Imaging

Mice were anesthetized as indicated above. When imaged for prolonged period of time, the isoflurane flow rate was reduced to 0.1 L/min. The dorsal skinfold window chamber was inserted onto a custom stabilization plate to prevent image motion artifacts and axial drifts over the time of the imaging session. Plane tracking to ensure that the same area is imaged repeatedly over the course of the drug uptake measurements was achieved through the use of a built-in Z-axis motor Animals were warmed with a heating plate in order to keep their temperature constant.

Green fluorescent microspheres (2.5 microns) (InSpeck, Invitrogen) were dried out using an EZ-2 evaporator (Genevac) and resuspended in sterile PBS. After sonication, the microspheres were then injected into the skin tissue of a dorsal window chamber on a nude mouse. Injections were performed with a CellTram vario (Molecular Devices) through pulled glass pipettes. After the skin tissue absorbed the PBS, images of the microspheres were taken at increasing depths. The vasculature in the window chamber was imaged under brightfield with a CCD camera using a 2× objective and overlaid with a fluorescence image using the same objective.

AZD2281-BODIPY FL (7.5 μl in DMSO) was mixed with 30 μl of 1:1 solutol:dimethylacetimide (Sigma) and slowly added to 112.5 microliters of PBS. The drug was injected through a tail vein intravenously and imaged with MFAM using a 25× objective. Confocal images of drug infusion into the tumor were taken using a 2× objective.

Image Processing

During image acquisition in two photon microscopy only a small number of photons are typically measured by the photodetectors with numbers ranging from tens to a few thousands with a statistical variation in the recorded number following a Poisson model of the noise. At lower counts per pixel, the error on the calculated anisotropy value will be then increasingly higher giving rise to images presenting severe noise artifacts. To account for noise induced variation we decided therefore to statistically weight every pixel anisotropy value within each image by its corresponding total intensity. Intensity weighted images were created by assigning colors based on anisotropy values, indicated by the scale bar, to each pixel in the fluorescence image. The intensity of the image is therefore dependent on the fluorescence intensity, while the color is dependent on the calculated anisotropy. In addition a BM3D collaborative filter was applied on each image.

Data Analysis

Images were analyzed in Matlab (Mathworks) and ImageJ. All anisotropy measurements were calculated from the equation r=(I∥−I⊥)/(I∥+2I⊥). The detector noise of the two photodetectors was subtracted from the whole images before the data were processed. Fluorescence images represent the denominator of the anisotropy equation, which represents the entire fluorescence from the sample. Anisotropy values were obtained by defining a region of interest and measuring the average anisotropy within that region. Regions were extended to fluorescent images to calculate the corresponding intensity. Regions were extended to fluorescent images to calculate the corresponding intensity.

Fluorescence Polarization

Fluorescence anisotropy measurements are based on the determination of the fluorescence polarization orientation with respect to that of the excitation light. During a photoselection process (FIG. 1A), only dipole-aligned fluorophores will have a high probability of getting excited by linear polarized light. Fluorophore emission will be aligned along the intrinsic emission dipole but Brownian motion will tend to induce loss of orientation and produce isotropic polarization emission. The degree of anisotropy is dictated by the correlation time τ□ defined by the Perrin equation, which is dependent on viscosity, size and temperature 2. To characterize the extent of linearly polarized emission, anisotropy, a dimensionless parameter r independent of emitted and excitation intensity (1) is then defined as the ratio of the polarized components to the total intensity.

r=(I₂−I₂)/(I₁+2I₂) (1)

When a fluorophore is free to rapidly rotate on a timescale that is shorter than its fluorescence lifetime (τ_{θ}<<τ), emission will be isotropic (depolarized). At high viscosity instead or when bound to larger molecules the rotational correlation time will increase (τ_{θ}>>τ) causing an anisotropy in the light distribution (FIG. 1A).

Fluorescence Lifetimes

The fluorescence anisotropy r is related to a fluorophore's lifetime τ_{f} and rotation correlation time τ_{σ} through the Perrin equation:

r₀/r=1+τ_{f}/τ_{θ} (2)

where r₀ is the fundamental anisotropy of the dye-dependent on the orientation of the excitation and emission dipole moments. To determine the contribution to the measured anisotropy values due to changes in fluorescence lifetime we measured the latest via fluorescence lifetime imaging microscopy (FLIM). For Me5-BODIPY in 0 or 90% glycerol solution we measured a fluorescent lifetime of 4.0±0.3 nsec for both conditions, indicating that the anisotropy dependence on glycerol concentration is caused by changes in the rotation correlation time τ_{θ}. The fluorescent microspheres (100% and 30%) had the same fluorescence lifetime, 3.6±0.3 nsec. Biotin-BODIPY FL also had similar values (4.0±0.3 nsec) when unbound or bound to NeutrAvidin. The change in anisotropy observed upon Avidin binding is therefore also due to changes in the rotation correlation time, caused by the large size of Avidin, and not due to a shift in fluorescence lifetime upon binding. AZD2281-BODIPY FL did demonstrate a subtle shift in fluorescence lifetime upon binding to PARP1 in vitro (4.1±0.3 nsec when unbound and 3.3±0.3 nsec when bound). Here, any contribution to anisotropy is likely minimal as the change in rotation correlation time is orders of magnitude bigger (unbound weight <1 kDa, bound to PARP1>120 kDa). However, when binding to smaller protein targets, any lifetime change will greatly influence anisotropy. Therefore FLIM could be considered as a complementary method to MFAM to elucidate the biophysical mechanism of anisotropy upon binding of fluorescent small molecules to larger protein targets.

Loss of Polarization Through Imaging

Light depolarization increases with increasing the objective's aperture angle. While depolarization is negligible under regular imaging conditions, it can play a role at high resolution and for single molecules studies. A change of 1.5% in the perpendicular component was previously observed for a 1.4 NA objective lens illuminated with linearly polarized light. For all our measurements a 1.05 NA objective lens was used and therefore we expect a smaller error.

In general the magnitude of the effects caused by the objective's numerical aperture is dependent on the specific objective used (magnification, numerical aperture), how the back aperture is filled (depolarization can be reduced by underfilling) together with proper alignment, and selected field of view. These effects are therefore strictly dependent on the particular setup. While compensation could be used through different calibration method 4, a restricted field of view guarantees that major artifacts are eliminated; center axis effects instead have a minimal impact. It's important to guarantee that all measurements are performed under controlled conditions maintaining constant the settings throughout our experiments to achieve reproducibility in values over time (FIG. 19).

Most importantly, by restricting the field of view, we ensured that polarization is uniform over the entire image as emphasized in FIGS. 2D, 2E. To accomplish uniformity we discarded data collected when the laser beam was scanned along the periphery of the objective (i.e. border of the images) because it could alter the anisotropy values over the field of view (and therefore the interpretation of the data). FIG. 2D shows anisotropy as calculated within the entire field of view compared with anisotropy calculated within the restricted field-of-view 3× (digital zooming), which was used for any measurement described herein.

Tissue Phantoms

The tissue optical phantoms used for characterization contained fluorescein (20 μM) (Sigma) which brought up in 1% Intralipid (10% Solution, Baxter Healthcare) in PBS with varying concentrations of India ink following a well established protocol. The corresponding scattering coefficient μ's was equal to 11 cm−1, a value typically considered for mouse tissue phantoms. Optical densities of ink concentrations in PBS were determined by measuring the absorbance spectrum at 910 nm. Fluorescent images of the solution were taken at 10-micron intervals through the depth of the phantoms.

During in vivo fluorescence imaging studies, tissue scattering of propagating photons combined with the spatial variation of tissue optical properties, affect, in a strong non-linear way, the intensity of the detected fluorescent photons. This effect could be detrimental when determining anisotropy values in the presence of low signal and/or for high values of anisotropy. Therefore, we measured optical phantoms with mouse-like tissue scattering properties (fixed) and with varying degrees of optical absorption to mimic the situation typically encountered when imaging in vivo or in vitro (different amount of fluorescent proteins expression, varying amount of signal depending on the absorption properties of the tissue (e.g. brain vs spleen), amount of bound fluorophore, etc), as shown in FIG. 16.

Embodiments of the biomedical system of the invention have been described as including a processor controlled by instructions stored in a memory. The memory may be random access memory (RAM), read-only memory (ROM), flash memory or any other memory, or combination thereof, suitable for storing control software or other instructions and data. Some of the functions performed by the discussed embodiments have been described with reference to flowcharts and/or block diagrams. Those skilled in the art should readily appreciate that functions, operations, decisions, etc. of all or a portion of each block, or a combination of blocks, of the flowcharts or block diagrams may be implemented as computer program instructions, software, hardware, firmware or combinations thereof. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

In addition, it is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

What is claimed is:

1. A system for spatially-resolving a portion of a target containing fluorescently-labeled target-bound molecules, of the fluorescently labeled molecules with the system comprising:
a source of light configured to generate light to be absorbed by the target via a multi-photon process;
an optical system positioned to optically relay light generated by the source of light onto an object plane of said system and form first and second images of said object plane, at first and second image planes respectively, in light emitted from the object plane
wherein said first image is formed in light emitted from the object plane and having only a first state of polarization;
wherein said second image is formed in light emitted from the object plane and having only a second state of polarization;
and
a processor programmed to transform said first and second images into a third image representing spatial anisotropy of said target.

2. A system according to claim 1, wherein said optical system includes
a microscope configured to epi-collect said light emitted from the object plane; and
a first optical detector positioned to receive said light emitted from the object plane and having only the first state of polarization; and
a second optical detector positioned to receive said light emitted form the object plane and having only the second state of polarization.

3. A system according to claim 2, wherein the processor is programmed to calculate a spatial distribution of anisotropy of the target according to r=(I₁−₂)/(I₁+2I₂), wherein r is a measure of said anisotropy, I₁ is the first image, and I₂ is the second image.

4. A system according to claim 1, wherein said optical system includes a microscope configured to collect said light emitted from the object plane in a confocal mode; and
a first optical detector positioned to receive said light emitted from the object plane and having only the first state of polarization; and
a second optical detector positioned to receive said light emitted form the object plane and having only the second state of polarization.

5. A method for a spatially-resolved optical detection of binding between a compound and a target, the method comprising:
optically imaging a combination of the a fluorescently labeled compound in the presence of the target to form an image representing a degree of anisotropy of light emitted by the combination; and
distinguishing a first portion of the target from a second portion of the target based on said image, the first portion being devoid of a target-bound compound, the second portion having the compound bound thereto.

6. A method according to claim 5, wherein the optically imaging includes imaging the combination in a competitive mode when an unlabeled compound is present to form an image representing a degree of anisotropy of light emitted by the combination;

7. A method according to claim 5, wherein said optically imaging includes collecting light from the combination with a microscopy system.

8. A method according to claim 5, wherein said optically imaging includes imaging of lifetime of fluorescence emitted by said fluorescently labeled compound

9. A method according to claim 5, wherein said optically imaging includes forming first and second images with first and second optical detectors, respectively, in fluorescent light emitted by the fluorescently labeled compound.

10. A method according to claim 9, further comprising causing the fluorescently labeled compound to generate the fluorescent light by exciting the fluorescently labeled compound with a multi-photon process.

11. A method according to claim 9, further comprising acquiring said fluorescent light having only a first state of polarization with the first optical detector, acquiring said fluorescent light having only a second state of polarization with the second optical detector.

12. A method according to claim 11, further comprising calculating spatial distribution of anisotropy of the target according to r=(I₁−I₂)/(I₁+2I₂), wherein r is a measure of said anisotropy, I₁ is the first image, and I₂ is the second image.

13. A method according to claim 5, wherein said distinguishing includes distinguishing first and second portions of a live cell.
